(19) United States
(12) Patent Application Publication
(10) Pub. No.: US 2020/0054654 A1
(43) Pub. Date: Feb. 20, 2020

(54) TOPICAL OLEAGINOUS COMPOSITIONS

(71) Applicant: DR. REDDY'S LABORATORIES LTD., Hyderabad (IN)

(72) Inventors: Sujit Kumar DOLAI, Brahmapur (IN); Basant AMARJI, Panna (IN); Pradip Kumar SASMAL, Hyderabad (IN); NV Anil Kumar RAVIPATI, Kistareddypet post (IN); Ujjawal BAIRAGI, Gautam Buddha Nagar (IN)

(21) Appl. No.: 16/531,969

(22) Filed: Aug. 5, 2019

(30) Foreign Application Priority Data
Aug. 16, 2018 (IN) 201841009717

(51) Int. Cl. A61K 31/69; A61K 31/381; A61K 31/436; A61K 31/335; A61K 47/14; A61K 9/00; A61K 47/44

(52) U.S. Cl. CPC A61K 31/69; A61K 31/381; A61K 31/436; A61K 47/44; A61K 47/14; A61K 9/0014; A61K 31/335

(57) ABSTRACT

A topical composition includes an oleaginous base and an active agent, which is useful for treating various skin disorders.

### FIELD OF THE INVENTION

The present application relates to a topical composition comprising an active agent and an oleaginous base. Further, the present application relates to a process of preparing such compositions and method of using such compositions in treating various skin disorder(s).

### BACKGROUND OF THE INVENTION

Topical compositions are widely used in the treatment of various skin conditions. Mainly, topical compositions that are oleaginous or emollient are useful in treating skin conditions that involve transdermal water loss, such as acne, dermatitis, psoriasis, and the like.

Inflammatory skin disorders are common worldwide. These inflammatory skin diseases include, for example, psoriasis, pityriasis rubra pilaris, pityriasis rosea, parapsoriasis, pityriasis lichenoides, lichen planus, lichen nitidus, erythema multiforme/Stevens-Johnson syndrome/toxic epidermal necrolysis, dermatitis herpetiformis, subcorneal pustular dermatosis, perioral dermatitis, allergic contact dermatitis, autosensitization dermatitis, Behcet's disease, acne vulgaris, rosacea, and atopic dermatitis.

Dermatitis is one of the inflammatory skin disorders that involve, for example, dry skin, increased transepidermal water loss, irritation, and pruritus. Dermatitis, as a condition, occurs as, for example, atopic dermatitis, contact dermatitis, and seborrheic dermatitis.

Atopic dermatitis (AD) or atopic eczema is a common skin disease that often begins in early childhood. The etiology of AD is likely multifactorial resulting from a complex interaction between genetic and environmental factors.

AD, also known as atopic eczema, is a type of inflammation of the skin (dermatitis). It results in itchy, red, swollen, and cracked skin. Clear fluid may come from the affected areas, which often thicken over time. Scratching worsens symptoms, and affected people have an increased risk of skin infections. The cause is unknown but believed to involve genetics, immune system dysfunction, and/or environmental exposures.

Topical steroids are widely used for inflammatory skin conditions like AD. For example, the following non-steroidal treatment options are available for treating mild to moderate atopic dermatitis: a) EUCRISA® (crisaborole 2%), b) PRUDOXIN® (Doxepin 5%), c) PROTOPIC® (tacrolimus 0.1%). However, there is a need for new and effective non-steroidal therapy for treating inflammatory skin disorder(s) like AD.

### SUMMARY OF THE INVENTION

The present application relates to a topical composition comprising an active agent and an oleaginous base.

An aspect of the present application relates to a topical composition comprising an active agent and an oleaginous base; wherein the oleaginous base is substantially free of water.

An aspect of the present application relates to a topical composition comprising an active agent and an oleaginous base; wherein the oleaginous base comprises an oleaginous vehicle in an amount of at least about 60% w/w based on the total weight of the composition.

An aspect of the present application relates to a topical composition comprising an active agent and an oleaginous base; wherein the oleaginous base comprises a skin penetration enhancer in an amount of less than about 20% w/w based on the total weight of the composition.

An aspect of the present application relates to a topical composition comprising an active agent and an oleaginous base; wherein the oleaginous base comprises a skin penetration enhancer and an oleaginous vehicle in an amount of at least about 60% w/w based on the total weight of the composition; wherein the skin penetration enhancer is in the form of a liquid at room temperature.

An aspect of the present application relates to a topical composition comprising an active agent and an oleaginous base; wherein the oleaginous base comprises (i) at least two stiffening agents and (ii) an oleaginous vehicle; wherein the stiffening agents selected from white wax, microcrystalline wax, emulsifying wax, cetyl esters wax, yellow wax, beeswax and any combination thereof, and the weight ratio between the two stiffening agents is in the range of from about 1:1 to about 3:1. In some embodiments, the weight ratio between the two stiffening agents is about 1:1, 3:2, 2:1, 5:2, 3:1, or 7:2.

An aspect of the present application relates to a topical composition comprising an active agent and an oleaginous base; wherein the oleaginous base comprises (i) one or more stiffening agent(s), (ii) a skin penetration enhancer, and (iii) an oleaginous vehicle having melting point more than about 35° C.; wherein the weight ratio between the skin penetration enhancer and the oleaginous vehicle is in the range of from about 1:5 to about 1:9.5. In some embodiments, the weight ratio between the skin penetration enhancer and the oleaginous vehicle is about 2:7, 1:5, 2:11, 1:6, 1:13, 1:7, 2:15, 1:8, 2:17, 1:9, 2:19, 1:10, 1:11, 1:12, 1:13, 1:14, 1:15, 1:16, 1:17, and 1:18.

An aspect of the present application relates to a topical composition comprising an active agent and an oleaginous base; wherein the oleaginous base comprises (i) one or more stiffening agent(s), (ii) a skin penetration enhancer, and (iii) an oleaginous vehicle. In some embodiments, the weight ratio between the stiffening agent(s) and the oleaginous vehicle is in the range of from about 1:45 to about 1:1. In some embodiments, the weight ratio between the stiffening agent(s) and the oleaginous vehicle is about 1:50, 1:45, 1:40, 1:35, 1:30, 1:25, 1:20, 1:15, 1:10, 1:5, or 1:1. In some embodiments, the weight ratio between the stiffening agent(s) and the skin penetration enhancer is in the range of from about 3:2 to about 1:1. In some embodiments, the weight ratio between the stiffening agent(s) and the skin penetration enhancer is about 3:1, 2:1, 3:2, 6:5, or 1:1.

An aspect of the present application relates to a topical composition comprising (a) an active agent, (b) one or more stiffening agent(s), (c) a skin penetration enhancer, (d) an oleaginous vehicle, and (e) a pharmaceutically acceptable excipient(s); wherein the composition comprises the stiffening agent, the skin penetration enhancer, and the oleaginous vehicle in a weight ratio from about 3:2:14 to about 2:1:17.

An aspect of the present application relates to a topical composition comprising (a) an active agent, (b) one or more stiffening agent(s), (c) a skin penetration enhancer, (d) an oleaginous vehicle having a melting point more than about 35° C., and (e) one or more pharmaceutically acceptable excipient(s); wherein the composition comprises the stiffening agent, the skin penetration enhancer, and the oleaginous vehicle in a weight ratio from about 3:2:14 to about 2:1:17. In some embodiments, the weight ratio between the stiffening agent(s), the skin penetration enhancer, and the oleaginous vehicle is about 2:1:13, 2:1:14, 2:1:15, 2:1:16, 2:1:17, 2:1:18, 3:2:13, 3:2:14, 3:2:15, 3:2:16, 3:2:17, or 3:2:18. In some embodiments, the weight ratio between the stiffening agent(s), the skin penetration enhancer, and the oleaginous vehicle is about 2:1:13, 2:1:14, 2:1:15, 2:1:16, 2:1:17, 2:1:18, 3:2:13, 3:2:14, 3:2:15, 3:2:16, 3:2:17, or 3:2:18.

The present application relates to a topical composition comprising (a) an active agent and (b) an oleaginous base; wherein the composition has an oleaginous base at least about 60% w/w based on the total weight of the composition, and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

Some aspects of the present application relates to a topical composition comprising (a) an active agent and (b) an oleaginous base; wherein the composition has an oleaginous base in an amount of at least about 70% w/w based on the total weight of the composition, and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

In some aspects of the present application, the topical composition provides release of active agent in a controlled manner.

In some aspects of the present application, the topical composition additionally comprises a water-miscible substance(s); wherein the weight ratio between the water-immiscible substance(s) to the water-miscible substance(s) is in the range from about 9:1 to about 9:0.1. In some embodiments, the weight ratio between the water-immiscible substance(s) and water-miscible substance(s) is about 9:1.1, 9:1, 9:0.9, 9:0.8, 9:0.7, 9:0.6, 9:0.6, 9:0.4, 9:0.3, 9:0.2, or 9:0.1.

In some aspects of the present application, the active agent is selected from a corticosteroid, a topical calcineurin inhibitor, antibiotic, anti-histamine, NSAID, COX-II inhibitor, antifungal, vitamin D or analogs, phosphodiesterase 4 (PDE4) inhibitor, 5-lipoxygenase inhibitor, retinoid compound, immunomodulator, and the like.

In some aspects of the present application, the active agent is selected from betamethasone, clobetasol, dexamethasone, mometasone, halobetasol, tretinoin, tazarotene, adapalene, tacrolimus, pimecrolimus doxepin, zileuton, cetirizine, diclofenac, ibuprofen, crisaborole, erythromycin, doxycycline, minocycline, celecoxib, mupirocin, miconazole, calcitriol, calcipotriene, retapamulin, chlorpheniramine and its pharmaceutically acceptable salts, prodrugs, esters, solvates, polymorphs thereof.

In some aspects of the present application, the active agent is selected from the group consisting of zileuton, crisaborole, doxepin, tacrolimus, and its pharmaceutically acceptable salts, solvates, esters, polymorphic forms, prodrugs, and combinations thereof.

In some aspects of the present application, the active agent is zileuton, its pharmaceutically acceptable salt, prodrug, ester, solvate, or polymorph thereof.

In some aspects of the present application, the skin penetration enhancer is substantially free of hydrophilic solvent(s).

In some aspects of the present application, the composition is substantially free of glycol(s).

In some aspects of the present application, the composition comprises glycol(s) lesser than the active agent's solubilizing levels.

In some aspects of the present application, the composition of the present application is occlusive and forms a thin oily film at the application site.

In some aspects of the present application, the composition is substantially anhydrous.

In some aspects of the present application, the active agent is a non-steroidal active agent.

In some aspects of the present application, the active agent is a non-steroidal active agent and is selected from the group consisting of tretinoin, tazarotene, adapalene, tacrolimus, pimecrolimus doxepin, zileuton, cetirizine, diclofenac, ibuprofen, crisaborole, erythromycin, doxycycline, minocycline, celecoxib, mupirocin, miconazole, calcitriol, calcipotriene, retapamulin, chlorpheniramine and its pharmaceutically acceptable salts, prodrugs, esters, solvates, polymorphs thereof and any combinations thereof.

In some aspects of the present application, the active agent is a non-steroidal active agent and is selected from the group consisting of zileuton, crisaborole, tacrolimus, doxepin, and its pharmaceutically acceptable salts, prodrugs, esters, solvates, polymorphs thereof and combinations thereof.

An aspect of the present application relates to a process of preparing topical oleaginous composition, the process comprising steps of a) preparing an oleaginous base by melting the oleaginous vehicle with one or more excipient(s), b) cooling the oleaginous base, c) addition of active agent to the oleaginous base with homogenization for at least 15 minutes to prepare the oleaginous composition, and d) cooling of the composition at the temperature of above about 40° C.±5° C. with stirring to prepare final composition.

An aspect of the present application relates to a method of treating and/or preventing skin disorder(s), by topically administering a composition comprising a therapeutically effective amount of an active agent to a subject in need thereof.

An aspect of the present application relates to a method of treating and/or preventing inflammatory skin disorder(s), by topically administering a composition comprising a therapeutically effective amount of a non-steroidal active agent to a subject in need thereof.

An aspect of the present application relates to a method of treating and/or preventing atopic dermatitis, by topically administering a composition comprising a therapeutically effective amount of a non-steroidal active agent to a subject in need thereof; wherein the non-steroidal active agent is selected from the group consisting of zileuton, crisaborole, tacrolimus, doxepin, and combinations thereof.

An aspect of the present application relates to a method of treating and/or preventing atopic dermatitis, by topically administering a composition comprising a therapeutically effective amount of zileuton, its pharmaceutically acceptable salt, prodrug, ester, solvate, or polymorph thereof.

An aspect of the present application relates to a method of treating and/or preventing pruritus, by topically administering a composition comprising a therapeutically effective amount of zileuton, its pharmaceutically acceptable salt, prodrug, ester, solvate, or polymorph thereof. In one aspect of the present application, the skin disorder is atopic dermatitis.

### BRIEF DESCRIPTION OF THE FIGURES

FIG. 1: Clinical skin severity scores of NC/Tnd mice treated with the topical test articles (Zileuton, PROTOPIC®, and vehicle).

FIG. 2: IVRT (In vitro release testing) studies on the oleaginous compositions of the present application

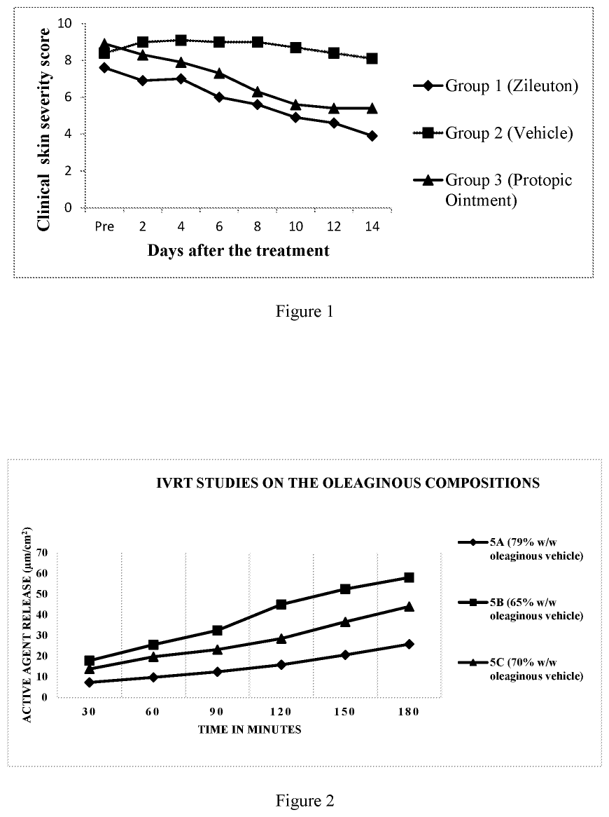

### DETAILED DESCRIPTION OF THE INVENTION

The details of one or more aspects of the presently-disclosed subject matter are outlined in this document. Modifications to aspects described in this document will be evident to those of ordinary skill in the art after a study of the information provided in this document. The information provided in this document, and particularly the specific details of the described exemplary aspects, is provided primarily for clearness of understanding and no unnecessary limitations are to be understood from there. In case of conflict, the specification of this document, including definitions, will control.

While the terms as used herein, are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a, “an,” and “the” as used herein, refers to “one or more” when used in this application, including the claims. Thus, for example, a reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters outlined in this specification and claims are approximations that can vary depending upon the desired properties sought to be obtained by the presently disclosed subject matter.

The term “about,” as used herein refers to a value or an amount of mass, weight, time, volume, concentration, temperature or percentage is meant to encompass variations of ±20% in some aspects, ±10% in some aspects, ±5% in some aspects, ±1% in some aspects, ±0.5% in some aspects, ±0.1% in some aspects, ±0.01% in some aspects, and ±0.001% in some aspects from the specified amount, as such variations are appropriate to perform the disclosed method. As used herein, ranges can be expressed as from “about” one particular value, and/or to “about” another particular value. It is also understood that there are many values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed. The term “about” in the context of ratio covers all decimal point of each numerical in the ratio. For example, about 1:2 will cover 1.2:2, 1.7:2, 1.9:2, 1:2.7, 1:2.9 and the other decimal variations.

The terms “applying,” “administering,” or “administration,” as used herein, refers to topical application of a zileuton composition applied or administered to affected and adjoining areas of skin by spreading or gentle rubbing or massaging.

The terms “active,” “active agent,” or “active substance,” as used herein, refers to a small molecule chemical substance that is used in the treatment of skin disorder(s). In some aspects of the present application, the active agent is specifically referred to one or more substance(s) from the group of betamethasone, clobetasol, dexamethasone, mometasone, halobetasol, tretinoin, tazarotene, adapalene, tacrolimus, pimecrolimus doxepin, zileuton, cetirizine, diclofenac, ibuprofen, crisaborole, erythromycin, doxycycline, minocycline, celecoxib, mupirocin, miconazole, calcitriol, calcipotriene, retapamulin, chlorpheniramine and its pharmaceutically acceptable salts, prodrugs, esters, solvates, or polymorphs thereof.

In some aspects of the present application, the active agent is one or more corticosteroids selected from betamethasone, clobetasol, halobetasol, dexamethasone, and their pharmaceutically acceptable salts prodrugs, esters, solvates, polymorphs thereof.

In some aspects of the present application, the active agent is a non-steroidal active agent.

In some aspects of the present application, the active agent is zileuton, its pharmaceutically acceptable salt, prodrug, ester, solvate, or polymorph thereof.

In some aspects of the present application, the active agent is crisaborole, its pharmaceutically acceptable salt, prodrug, ester, solvate, or polymorph thereof.

In some aspects of the present application, the active agent is doxepin, its pharmaceutically acceptable salt, prodrug, ester, solvate, or polymorph thereof.

In some aspects of the present application, the active agent is tacrolimus, its pharmaceutically acceptable salt, prodrug, ester, solvate, or polymorph thereof.

The term “base composition” as used herein, refers to a pharmaceutical composition which does not contain any active agent(s). In some aspects of the present application, the base composition may be in the form of monophasic or biphasic and can be selected from an oleaginous base, an emulsion base, and an ointment base composition.

The term “composition” or “formulation” as used herein, refers to preparation for delivering effective amounts of an active agent locally to the mammal. The compositions of the present application, without any limitation, may be present in therapeutic dosage forms like, transdermal or topical dosage form such as lotion, ointment, spray, aerosol, emulsion, paste, suspension, foam, cream, gel, and the like; and the composition may be administered in any suitable routes or any combinations with or without device(s) thereof. In an aspect of the present application, the composition is topically administered to treat a skin disorder, and it is preferably a semi-solid dosage form.

The term “effective amount” or “therapeutically effective amount” as used herein, refers to a concentration of the active agent in the composition which is sufficient to achieve the intended purpose as compared to patients treated with the vehicle. This can vary depending on the patient, the condition, and the treatment being effected. The exact amount that is required will vary from subject to subject, depending on the species, age, and general condition of the subject, the particular carrier or adjuvant being used, mode of administration, and the like. As such, the effective amount will vary based on the particular circumstances and such an amount can be determined in a particular case by one of ordinary skill in the art using only routine experimentation.

The term “subject” as used herein, refers to any mammal such as human, rat, mouse, monkey, and the like. In an aspect of the present application, the subject is human. The term “subject” can be interchangeably used with the term “patient.” In an aspect of the present application, the subject is suffering from an inflammatory skin disorder. The inflammatory skin disorder is selected from the group of acne, psoriasis, allergic dermatitis, pruritus, atopic dermatitis, allergic dermatitis, seborrheic dermatitis, contact dermatitis, erythema, eczema, and the like.

The term “topical composition” as used herein, refers to a topical composition that comprises an active agent.

The term “related substances” or “impurities” mean the degradation impurities formed in the composition during shelf life or active ingredient's process-related impurities of drug materials.

The term “stability” or “stable” as used herein, includes both chemical stability and physical stability. The term “stability” is defined as the ability of a drug substance or drug product to remain within the established specifications to maintain its identity, strength, quality, and purity at least until its expiration date. The term ‘chemical stability’ means the tendency of the drug to resist changes or decomposition due to chemical reactions, or due to the effects of oxygen, heat, light, pressure, etc. The term “physical stability” refers to maintaining the physical and polymorphic form of the active agents, such as crystalline, amorphous, or mixtures thereof, and “chemical stability” refers to maintaining acceptable concentrations of drug-related impurities.

The term “room temperature” as used herein, means any temperature point above about 5° C.±3° C. In some aspects of the present application, the room temperature means any temperature point selected between about 5° C. and about 35° C.

The terms “excipient” or “topically acceptable excipient” or “pharmaceutically acceptable excipient” or “dermatologically acceptable excipient” are used interchangeably to mention any excipient which is acceptable for using in topical compositions and does not provide any therapeutic effect, and may contribute to aesthetic properties or any relevant non-therapeutic function of the topical composition.

The term “substantially free” as used herein, refers to absence or presence to some marginal extent, such as in an amount of about 0% to in an amount of less than about 10%. In some aspects of the present application, the term “substantially free” as used herein, indicates that the specified substance referred to is present in amounts less than about 10% by weight of the total composition or in an amount of less than about 9% by weight of the total composition, or in an amount of less than about 8% by weight of the total composition, or in an amount of less than about 7% by weight of the total composition, or in an amount of less than about 6% by weight of the total composition, or in an amount of less than about 5% by weight of the total composition, or in an amount of less than about 4% by weight of the total composition, or in an amount of less than about 3% by weight of the total composition, or in an amount of less than about 2% by weight of the total composition, or in an amount of less than about 1% by weight of the total composition, or in an amount of less than about 0.01% by weight of the total composition, or in an amount of less than about 0.001% by weight of the total composition, or in an amount of about 0% by weight of the total composition or completely free of specified substance (i.e.) 0%. If the term “substantially free” is used before the active agent or related substance(s), then it refers to in an amount of less than 10% based on the total amount of active agent, not based on the total weight of the composition.

The term “non-solubilized” as used herein, refers to approximately in an amount of about 90% of the specified substance in the non-solubilized form in the composition, meaning the specified substance is dispersed in the composition, or that a negligible amount is present in the solubilized form, i.e., in an amount of less than about 10% of the specified substance may be solubilized or degraded or exist in some other form in the composition. For example, zileuton is in non-solubilized form in the composition means in an amount of more than about 90% or in an amount of more than about 91% or in an amount of more than about 92% or in an amount of more than about 93% or in an amount of more than about 94% or in an amount of more than about 95% or in an amount of more than about 96% or in an amount of more than about 97% or in an amount of more than about 98% or in an amount of more than about 99% or in an amount of about 100% of total zileuton is dispersed in the composition, and remaining amount of zileuton may exist in the form of solubilized or degraded to related substance(s), or exist in some other form.

The term “oleaginous base” as used herein, refers to an oleaginous composition that comprises one or more water-immiscible substance(s) in an amount of at least about 60% w/w based on the total weight of the composition. The oleaginous base may comprise a mixture of water-immiscible substance(s) that are from liquid, solid, or semi-solid water-immiscible sub stance(s).

In some aspects of the present application, the oleaginous base may be in the form of water-in-oil emulsion, glycol/water-in-oil emulsion, glycol-in-water emulsion, or ointment base.

In an aspect of the present application, the topical composition is not an oil-in-water emulsion.

In some aspects of the present application, the oleaginous base comprises in an amount of at least about 60% or in an amount of at least about 61% or in an amount of at least about 62% or in an amount of at least about 63% or in an amount of at least about 64% or in an amount of at least about 65% or in an amount of at least about 66% or in an amount of at least about 67% or in an amount of at least about 68% or in an amount of at least about 69% or in an amount of at least about 70% or in an amount of at least about 71% or in an amount of at least about 72% or in an amount of at least about 73% or in an amount of at least about 74% or in an amount of at least about 75% or in an amount of at least about 76% or in an amount of at least about 77% or in an amount of at least about 78% or in an amount of at least about 79% or in an amount of at least about 80% or in an amount of at least about 81% or in an amount of at least about 82% or in an amount of at least about 83% or in an amount of at least about 84% or in an amount of at least about 85% or in an amount of at least about 86% or in an amount of at least about 87% or in an amount of at least about 88% or in an amount of at least about 89% or in an amount of at least about 90% or in an amount of at least about 91% or in an amount of at least about 92% or in an amount of at least about 93% or in an amount of at least about 94% or in an amount of at least about 95% or in an amount of at least about 96% or in an amount of at least about 97% or in an amount of at least about 98% or in an amount of at least about 99% or in an amount of at least about 100% of one or more water-immiscible substance(s) based on the total weight of the oleaginous base.

The water-immiscible substance is selected from one or more stiffening agent(s), an oleaginous base, liquid oil substance, a skin penetration enhancer, and/or an oleaginous vehicle.

The term “oleaginous vehicle” as used herein, refers to an inactive excipient(s) that is a water-immiscible substance, or mixture of more than one water-immiscible substance(s) and is present in the composition of the present application in larger amount than any other excipient(s). In some aspects of the present application, the oleaginous vehicle refers to one or more water-immiscible substance(s) having a melting point more than about 35° C.

The term “pharmaceutically acceptable salts” as used herein, refers to an active agent that is obtained by reacting active agent(s) with acids, inorganic bases, organic bases, compounds having acid group, alkaline earth metal salts, amino acids. The pharmaceutically acceptable salts will retain the therapeutic effectiveness and properties of the active agent(s). In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salts are not specifically limited as far as it can be used in medicaments. Examples of a salt that the compounds described herein form with a base include the following: salts thereof with inorganic bases such as sodium, potassium, magnesium, calcium, and aluminium; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid; organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

In some aspects of the present application, the pharmaceutically acceptable salts also encompass the acid addition salts, solvate, prodrugs, and polymorphic forms of the given active agent.

The present application relates to a topical composition comprising an active agent and an oleaginous base.

In an aspect of the present application, the composition further comprises one or more pharmaceutically acceptable excipient(s) selected from preservative, polymer, stiffening agent, water, water-miscible substance, water-immiscible substance, emollient, solvent, skin penetration enhancer, surfactant, pH modifying agent, antioxidant, and combinations thereof.

The term “preservative” as used herein, refers to, but are not limited to, a natural or synthetic chemical that prevents the decomposition of the composition by microbial growth or by undesirable chemical changes. Preservatives can desirably be incorporated into a composition for protecting against the growth of potentially harmful microorganisms while microorganisms tend to grow in an aqueous phase and can also reside in a hydrophobic or oil phase. Examples of preservatives that can be used in the present application include, but are not limited to, methylparaben, propylparaben, phenoxyethanol, benzyl alcohol, chlorocresol, benzalkonium chloride, cetrimonium chloride, sodium edetate, disodium edetate, boric acid, sorbic acid, or any mixtures thereof.

The term “polymer” as used herein, refers to but are not limited to, carbomers, colloidal silicon dioxide, cellulose and derivatives such as cellulose, ethylcellulose, methylcellulose, carboxymethyl hydroxyethylcellulose, cellulose acetate propionate carboxylate, hydroxyethylcellulose, hydroxyethyl ethylcellulose, hydroxypropyl cellulose, hydroxypropyl methylcellulose, methyl hydroxyethylcellulose, hydroxybutyl methylcellulose, microcrystalline cellulose, sodium cellulose sulfate, cellulose acetate phthalates, cellulose acetate butyrates, hydroxypropyl methyl cellulose phthalates and mixtures thereof. The term “polymer” or “polymeric substance” does not cover hydrocarbons, petrolatum, paraffin, cyclomethicone, siloxane, silicone substances, resins, and the like.

The terms “thickening agent” or “stiffening agent” or “gelling agent” or “solidifying agent” are used interchangeably to mean a substance which increases the thickness or hardness or viscosity of the composition and to give bulkiness to the composition.

Stiffening agents that may be used in the present application include carbomers, colloidal silicon dioxide, cellulose and derivatives such as cellulose, ethylcellulose, methylcellulose, carboxymethyl hydroxyethylcellulose, cellulose acetate propionate carboxylate, hydroxyethylcellulose, hydroxyethyl ethylcellulose, hydroxypropyl cellulose, hydroxypropyl methylcellulose, methyl hydroxyethylcellulose, hydroxybutyl methylcellulose, microcrystalline cellulose, sodium cellulose sulfate, cellulose acetate phthalates, cellulose acetate butyrates, hydroxypropyl methylcellulose phthalates and mixtures thereof. Other useful thickeners include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, sclerotium gum, sodium carboxymethyl dextran, sodium carrageenan, tragacanth gum, xanthan gum, synthetic and natural glues, polymeric resins, cetyl alcohol, cetyl esters wax, paraffin, stearyl alcohol, lauryl alcohol, cetostearyl alcohol, white wax, yellow wax, beeswax, white beeswax, candelilla wax, emulsifying wax, cotton wax, carnauba wax, bayberry wax, rice-bran wax, hard fat, cetyl palmitate, hard paraffin, myristyl alcohol, ceresin wax and mixtures thereof. Also useful are acrylic acid/ethyl acrylate copolymers and the carboxyvinyl polymers under the trademark of Carbopol resins. Other examples include Sepineo™ P 600, Carbopol® products, PEG 400, Eudragit® 100, Eudragit® RSPO, Eudragit® RLPO, Eudragit® ND40, Plasdone®, Dry-FLO (aluminum starch octenyl succinate), copolymers based on butyl methacrylate and methyl methacrylate (Plastoid® B).

Suitable stiffening agents may include waxy materials such as candelilla, carnauba, beeswax, spermaceti, carnauba, bayberry, montan, ozokerite, ceresin wax, cetyl ester wax, paraffin, synthetic waxes such as Fisher-Tropsch waxes, silicone waxes, microcrystalline waxes and the like; soaps, such as the sodium and potassium salts of higher fatty acids, i.e., acids having from 12 to 22 carbon atoms; amides of higher fatty acids; higher fatty acid amides of alkylolamines; dibenzaldehyde-monosorbitol acetals; alkali metal and alkaline earth metal salts of the acetates, propionates and lactates; and mixtures thereof.

The term “solvent” as used herein, refers to a substance that is used to solubilize the active agent in the composition. In some aspects of the present application, the term “solvent” is used to herein can be interchangeably used to refer to a skin penetration enhancer, a solubilizer, a liquid water-immiscible substance, and the like.

The term “surfactant” or “emulsifying agent” as used herein, refers to a chemical substance that is amphiphilic and capable of forming emulsion composition. In an aspect of the present application, the surfactant is selected from anionic, cationic, non-ionic, and amphoteric surfactant(s).

Anionic surfactants dissociate in water into an amphiphilic anion and a cation (usually an alkali metal or ammonia). The amphiphilic portion generally contains an acid, sulfate, or sulfonate group, which bears the negative charge. Anionic surfactants used in pharmaceutical preparations include alkali-metal soaps (monovalent alkyl carboxylates) which are the sodium and potassium salts of the higher fatty acids. They are often produced from vegetable oils or specific fatty acids such as stearic acid, lauric acid, or oleic acid; animal fats such as tallow may also be used. Ammonium soaps have similar properties. Metallic soaps (polyvalent alkyl carboxylates), the calcium, zinc, magnesium, and aluminum salts of the higher fatty acids may also be used. Amine soaps, which are the amine salts of fatty acids and include trolamine (triethanolamine) stearate and diolamine (diethanolamine) stearate, may also be used.

Cationic surfactants are used alone or in combination with another emulsifying agent(s).

Nonionic surfactants glycol and glycerol esters (monoesters of ethylene glycol, diethylene glycol, and propylene glycol, and mono- or diesters of glycerol) contain both ester and hydroxyl groups and are widely used as non-ionic surfactants. Macrogol esters: Polyethoxylation of glycols provides additional hydrophilicity, which increases with the degree of ethoxylation, and fatty acid esters with a wide range of macrogols (polyethylene glycols) are used. Glycol ethers: Ethers of glycols with fatty alcohols are generally included in the same class as macrogol ethers and are used similarly. Macrogol ethers: Ethers of macrogols with fatty alcohols (macrogol alkyl ethers) or alkylphenols (macrogol aryl ethers) have similar properties to macrogol esters, but the ether linkage is more stable to hydrolysis making macrogol ethers more resistant to acids and alkalis. Polyalcohol esters: Fatty acid esters of polyalcohols such as glycerol polymers (polyglycerols), sorbitol, and sucrose also have nonionic surfactant properties. Sorbitan esters (esters of the cyclic mono- or di-anhydrides of sorbitol with fatty acids) are oil-soluble, water-dispersible, nonionic surfactants and are effective water-in-oil emulsifiers. Polysorbates (polyethoxylated sorbitan esters) are more hydrophilic, water-soluble compounds and are used as oil-in-water emulsifying agents. Poloxamers are copolymers of polyoxyethylene and polyoxypropylene.

Examples of non-ionic surfactants are selected from, but are not limited to, acetoglycerides, diethylene glycol esters, diethylene glycol ethers, ethylene glycol esters, glyceryl behenate, glyceryl mono- and di-esters, glyceryl monocaprylocaprate, glyceryl monolinoleate, glyceryl mono-oleate, glyceryl stearates, macrogol cetostearyl ethers, macrogol/glycerol esters, macrogol 6 glyceryl caprylocaprate, macrogol 20 glyceryl monostearate, macrogol 15 hydroxystearate, macrogol laurates, macrogol lauryl ethers, macrogol monomethyl ethers, macrogol oleates, macrogol oleyl ethers, macrogol 40 sorbitol heptaoleate, macrogol stearates, macrogolglycerol cocoates, nonoxinols, octoxinols, oleyl oleate, palmitic acid, poloxamers, polyoxyl castor oils, polyoxyl hydrogenated castor oils, polysorbates, polyvinyl alcohol, propylene glycol caprylates, propylene glycol diacetate, propylene glycol laurates, propylene glycol monopalmitostearate, quillaia, sorbitan esters, sucrose esters, triglycerol diisostearate, tyloxapol. Glycol and glycerol esters are selected from glyceryl behenate, glyceryl mono- and di-esters, glyceryl monocaprylocaprate, glyceryl monolinoleate, glyceryl mono-oleate, glyceryl distearate, glyceryl monostearate, glyceryl palmitostearate, diethylene glycol esters such as diethylene glycol monolaurate, diethylene glycol mono-oleate, diethylene glycol monostearate, diethylene glycol palmitostearate, ethylene glycol esters such as ethylene glycol distearate, ethylene glycol monopalmitostearate, propylene glycol esters such as propylene glycol dicaprylocaprate, propylene glycol monocaprylate, propylene glycol diacetate, propylene glycol dilaurate, propylene glycol monolaurate, propylene glycol monopalmitostearate, glycol ethers diethylene glycol ethers such as diethylene glycol monoethyl ether, macrogol derivatives such as ethoxylated glycerol esters, macrogol-6-glyceryl caprylocaprate, macrogol 20 glyceryl monostearate, macrogolglycerol cocoates, polyoxyl 35 castor oil, polyoxyl 40 hydrogenated castor oil, macrogol esters such as macrogol 15 hydroxystearate, macrogol laurates, macrogol oleates, macrogol stearates, macrogol/glycerol esters like behenoyl macrogolglycerides, caprylocaproyl macrogolglycerides, lauroyl macrogolglycerides, linoleoyl macrogolglycerides, oleyl macrogolglycerides, stearoyl macrogolglycerides, macrogol alkyl ethers such as macrogol lauryl ethers, macrogol monomethyl ethers, macrogol oleyl ethers, macrogol aryl ethers such as nonoxinol 9, nonoxinol 10, nonoxinol 11, octoxinol 9, octoxinol 10, tyloxapol; polyalcohol esters such as polyglycerol esters, triglycerol diisostearate, sorbitan esters such as sorbitan laurate, sorbitan oleate, sorbitan palmitate, sorbitan sesquioleate, sorbitan stearate, sorbitan trioleate, sorbitan tristearate, sorbitan macrogol esters such as macrogol 40 sorbitol heptaoleate, polysorbate 20, polysorbate 40, polysorbate 60, polysorbate 80, polysorbate 85, sucrose esters, poloxamers such as poloxalene, poloxamer 188, poloxamer 407.

The surfactant is, but are not limited to, disodium cocoamphodiacetate, oxyethylenated glyceryl cocoate (7 EO), PEG-20 hexadecenyl succinate, PEG-15 stearyl ether, ricinoleic monoethanolamide monosulfosuccinate salts, oxyethylenated hydrogenated ricinoleic triglyceride containing 60 ethylene oxide units such as the products marketed by BASF under the trademarks CREMOPHOR® RH 60 or CREMOPHOR® RH 40 (polyoxyl 40 hydrogenated castor oil), polymers such as poloxamers, which are block copolymers of ethylene oxide and propylene oxide, and the nonsolid fatty substances at room temperature (that is to say, at temperatures ranging from about 20 to 35° C.) such as sesame oil, sweet almond oil, apricot stone oil, sunflower oil, octoxyglyceryl palmitate (or 2-ethylhexyl glyceryl ether palmitate), octoxyglyceryl behenate (or 2-ethylhexyl glyceryl ether behenate), dioctyl adipate, and tartrates of branched dialcohols. Sorbitan fatty acid esters are a series of mixtures of partial esters of sorbitol and its mono- and dianhydrides with fatty acids. Sorbitan esters include products marketed as ARLACEL® 20, ARLACEL® 40, ARLACEL® 60, ARLACEL® 80, ARLACEL® 83, ARLACEL® 85, ARLACEL® 987, ARLACEL® C, PEG-6 stearate and glycol stearate and PEG-32 stearate (TEFOSE® 63), and PEG-6 stearate and PEG-32 stearate (TEFOSE® 1500), glyceryl stearate and PEG 100 stearate (TEFOSE® 165) and any mixtures thereof. Polyethylene glycol ethers of stearic acid are in another group of emulsifiers that can be used in the emulsions. Examples of polyethylene glycol ethers of stearic acid include, but are not limited to, steareth-2, steareth-4, steareth-6, steareth-7, steareth-10, steareth-11, steareth-13, steareth-15, steareth-20, polyethylene glycol ethers of stearyl alcohol (steareth 21), and any mixtures thereof. Other emulsifying agents include sodium lauryl sulphate, cetyl trialkyl ammonium bromide, polyoxyethylene sorbitan fatty acid esters, and any mixtures thereof.

Nonionic surfactant(s) include those that can be broadly defined as condensation products of long-chain alcohols, e.g., C8-30 alcohols, with sugar or starch polymers, i.e., glycosides. Various sugars include, but are not limited to, glucose, fructose, mannose, and galactose, and various long-chain alcohols include, but are not limited to, decyl alcohol, cetyl alcohol, stearyl alcohol, lauryl alcohol, myristyl alcohol, oleyl alcohol, and any mixtures thereof.

Other useful nonionic surfactants include condensation products of alkylene oxides with fatty acids such as alkylene oxide esters of fatty acids. Other nonionic surfactants are the condensation products of alkylene oxides with 2 moles of fatty acids such as alkylene oxide diesters of fatty acids.

Examples of amphoteric and zwitterionic surfactants include those which are broadly described as derivatives of aliphatic secondary and tertiary amines in which the aliphatic radical can be straight or branched chain; wherein one of the aliphatic substituents contains from about 8 to about 22 carbon atoms, and one contains an anionic water-solubilizing group, e.g., carboxy, sulfonate, sulfate, phosphate, or phosphonate.

Silicone surfactants are typically organically modified organopolysiloxanes, sometimes called silicone surfactants. Useful silicone emulsifying agents include dimethiconle copolyols. These materials are polydimethyl siloxanes, which have been modified to include polyether side chains such as polyethylene oxide chains, polypropylene oxide chains, mixtures of these chains, and polyether chains containing moieties derived from both ethylene oxide and propylene oxide.

Co-emulsifiers or secondary surfactants include, but are not limited to, polyoxylglycerides such as oleoyl macrogolglycerides (LABRAFIL® M 1944CS), linoleoyl macrogolglycerides (LABRAFIL® M 2125CS), caprylocaproyl macrogolglycerides (LABRASOL®), cetyl alcohol (and) ceteth-20 (and) steareth-20 (EMULCIRETM 61 WL 2659), glyceryl stearate (and) PEG-75 stearate (GELOT® 64), d-alpha tocopheryl polyethylene glycol 1000 succinate (TPGS) and any mixtures thereof.

The term “oil” as used herein, refers to one or more water-immiscible substances. In an aspect of the present application, the oil substance is liquid water-immiscible substance(s) i.e. liquid at room temperature, selected from isopropyl myristate, isopropyl palmitate, oils of natural origin such as almond oil, coconut oil, olive oil, palm oil, peanut oil and the like, fatty acids such as lauric acid, myristic acid, palmitic acid, and stearic acid, monohydric alcohol esters of the fatty acids such as ethyl laurate, isopropyl laurate, ethyl myristate, n-propyl myristate, isopropyl myristate, ethyl palmitate, isopropyl palmitate, methyl palmitate, methyl stearate, ethyl stearate, isopropyl stearate, butyl stearate, isobutyl stearate, amyl stearate, isoamyl stearate, branched or linear long-chain aliphatic alcohols such as lauryl alcohol, myristyl alcohol, and stearyl alcohol, or mixtures thereof. Exemplary emollients include caprylic/capric triglycerides, castor oil, ceteareth-20, ceteareth-30, cetearyl alcohol, ceteth 20, cetostearyl alcohol, cetyl alcohol, cetyl stearyl alcohol, cocoa butter, diisopropyl adipate, glycerin, allantoin, glyceryl monooleate, glyceryl monostearate, glyceryl stearate, isopropyl myristate, isopropyl palmitate, lanolin, lanolin alcohol, hydrogenated lanolin, liquid paraffins, white soft paraffin, linoleic acid, mineral oil, oleic acid, white petrolatum, silicones and mixtures thereof.

The term “emollients” as used herein, refers to substances that soften and soothe the skin. They are used to prevent dryness and scaling of the skin. Examples of emollients that can be used in the present application include, but are not limited to, oils of natural origin such as almond oil, coconut oil, olive oil, palm oil, peanut oil and the like, fatty acids such as lauric acid, myristic acid, palmitic acid, and stearic acid, monohydric alcohol esters of the fatty acids such as ethyl laurate, isopropyl laurate, ethyl myristate, n-propyl myristate, isopropyl myristate, ethyl palmitate, cetyl palmitate, isopropyl palmitate, methyl palmitate, methyl stearate, ethyl stearate, isopropyl stearate, butyl stearate, isobutyl stearate, amyl stearate, and isoamyl stearate, glycols such as ethylene glycol, diethylene glycol, polyethylene glycol, branched aliphatic alcohols such as lauryl alcohol, myristyl alcohol, and stearyl alcohol, or mixtures thereof. Exemplary emollients include caprylic/capric triglycerides (medium-chain triglycerides), castor oil, ceteareth-20, ceteareth-30, cetearyl alcohol, ceteth 20, cetostearyl alcohol, cetyl alcohol, cetyl stearyl alcohol, cocoa butter, diisopropyl adipate, glycerin, PPG-15 stearyl ether, glyceryl monooleate, glyceryl monostearate, glyceryl stearate, isopropyl myristate, isopropyl palmitate, lanolin, lanolin alcohol, hydrogenated lanolin, lanolin derivatives, cholesterol, liquid paraffins, linoleic acid, mineral oil, oleic acid, isostearyl neopentanoate, octyl stearate, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, white petrolatum, polyethylene glycol, polyoxyethylene glycol fatty alcohol ethers, glyceryl tricaprylate, silicones and mixtures thereof. The water-miscible emollient(s) are selected from the group comprising of glycerol, sorbitol, octyl dodecanol, ethylene glycol, polyethylene glycol (2 to 20 monomers), propylene glycol, dipropylene glycol, butylene glycol, pentylene glycol, and hexylene glycol.

In one of the aspect of the present application, the emollient(s) are liquid water-immiscible(s) substance selected from group consisting of one or more water-immiscible substance(s) selected from fatty alcohol(s), fatty acid(s), ethers of fatty alcohol(s), esters of fatty acid(s), terpenes, mineral oil, soft paraffin, hard paraffin, petrolatum, mixture of mineral oil and lanolin alcohols, coconut oil, almond oil, lanolin, mixture of petrolatum and lanolin alcohols, vegetable oils, and mixtures thereof.

In an aspect of the present application, the emollients or a liquid water-immiscible substance is selected from isopropyl myristate, medium-chain triglyceride, stearic acid, myristyl alcohol, oleic acid, oleyl alcohol, octyl dodecanol, mineral oil, paraffin, liquid paraffin, almond oil, dibutyl sebacate, limonene, cetyl ester wax, isopropyl palmitate, cyclomethicone, ceresin wax, cetyl ester wax, glyceryl tricaprylate; tricaprylin, propylene Glycol monolaurate, octanoic acid, octyldodecyl myristate, Isostearyl alcohol, oleyl oleate, PPG-15 stearyl ether, and any combinations thereof.

The term “antioxidants” as used herein, refers to substances which inhibit oxidation or suppress reactions promoted by oxygen or peroxides. Antioxidants, especially lipid-soluble antioxidants, can be absorbed into the cellular membrane to neutralize oxygen radicals and thereby protect the membrane. Suitable antioxidants that can be used in the present application include, but are not limited to, citric acid monohydrate, ascorbic acid (vitamin C), glutathione, sodium metabisulfite, lipoic acid, uric acid, sorbic acid, carotenes, alpha-tocopherol (vitamin E), TPGS, ubiquinol, butylated hydroxyanisole, butylated hydroxytoluene, sodium benzoate, propyl gallate (PG, E310), and tertiary-butylhydroquinone.

The term “pH modifying agent” as used herein, refers to an organic or inorganic chemical substance used for adjusting pH of the composition, is selected from weak organic acid, weak inorganic acids, bases, alkaline substance(s), and the like. The pH modifying agent is selected from, but are not limited to, bases such as calcium hydroxide, sodium hydroxide, potassium hydroxide; amines such as triethanolamine; acids such as citric acid, lactic acid, hydrochloric acid.

The topical composition of the present application comprises one or more fatty alcohol(s). Examples of fatty alcohol are saturated or unsaturated aliphatic alcohol having 8 to 25 carbon atoms, a linear or branched, saturated or unsaturated aliphatic alcohol, but are not limited to, behenyl alcohol, cetostearyl alcohol, oleyl alcohol, cetyl alcohol, isocetyl alcohol, isostearyl alcohol, lauryl alcohol, myristyl alcohol, stearyl alcohol, C30-50 alcohols, and lanolin alcohol.

An aspect of the present application relates to a topical composition comprising an active agent and an oleaginous base; wherein the oleaginous base comprises one or more water-immiscible substance(s).

An aspect of the present application relates to a topical composition comprising an active agent and an oleaginous base; wherein the oleaginous base comprises an oleaginous vehicle in an amount of at least about 60% w/w based on the total weight of the composition.

In some aspects of the present application, the oleaginous base comprises one or more solid water-immiscible substances having a melting point more than about 35° C., and one or more liquid water-immiscible substance.

An aspect of the present application relates to a topical composition comprising (a) an active agent, (b) an oleaginous base comprising one or more stiffening agent(s), and c) one or more pharmaceutically acceptable excipient(s).

An aspect of the present application relates to a topical composition comprising a) an active agent b) an oleaginous base comprising one or more stiffening agent(s), and c) one or more pharmaceutically acceptable excipient(s); wherein the active agent is present in a non-solubilized form in the composition.

In some aspects of the present application, the oleaginous base comprises (i) one or more stiffening agent(s), (ii) a skin penetration enhancer and (iii) an oleaginous vehicle having a melting point more than about 35° C.; wherein the skin penetration enhancer is liquid at room temperature and the skin penetration enhancer is selected from lower alcohols, glycol, glycol esters, glycol ethers, fatty acids, fatty alcohols, fatty acid esters, medium-chain triglycerides, terpenes, alkanones, sulfoxides, nitrogenous compounds, isosorbide derivatives and combinations thereof.

A “permeation enhancer” or “skin penetration enhancer” as used herein, refers to enhance the penetration rate of the drugs through the skin or mucous membrane. Permeation enhancers have also been called “accelerants” and “absorption promoters.” In some aspects of the present application, the skin penetration enhancers, is one or more water-immiscible substance(s), and the water-immiscible penetration enhancers are selected from the group comprising, but are not limited to, fatty alcohol(s), fatty acid(s), ethers of fatty alcohol(s), esters of fatty acid(s), terpenes, mineral oil, soft paraffin, hard paraffin, petrolatum, mixture of mineral oil and lanolin alcohols, coconut oil, almond oil, lanolin, mixture of petrolatum and lanolin alcohols, vegetable oils, and mixtures thereof. The fatty alcohol(s) are selected from but are not limited to, stearyl alcohol, isostearyl alcohol, linolenyl alcohol, octyl dodecanol, oleyl alcohol, lauryl alcohol, behenyl alcohol, and the like. The fatty acid(s) are selected from, but are not limited to, oleic acid, isostearic acid, lauric acid, myristic acid, n-octanoic acid, palmitic acid, stearic acid, and the like. The ethers of fatty alcohol(s) are selected from stearyl alcohol ethers such as polypropylene glycol 15 stearyl ethers and the like. The esters of fatty acid(s) are selected from, but are not limited to, ethyl oleate, polyglyceryl-3 dioleate triglycerides of oleic acid, triglycerides of caproic acid, diisopropyl adipate, octyl dodeconol, dibutyl sebacate, diisopropyl sebacate, isopropyl myristate, isopropyl palmitate, medium-chain triglycerides, methyl propionate and the like. The vegetable oils selected from, but are not limited to, almond oil, coconut oil, corn oil, cottonseed oil, linseed oil, oil of mink, olive oil, palm oil, sunflower oil, nut oil and the like.

In some aspects of the present application, the skin penetration enhancer is one or more fatty acid ester(s). Fatty acid esters are a type of ester that results from the combination of a fatty acid with alcohol; examples being diisopropyl sebacate, dibutyl sebacate, isopropyl myristate, isopropyl palmitate, methyl propionate, and any mixture thereof. Isopropyl myristate is the ester of isopropyl alcohol and myristic acid.

An aspect of the present application relates to a topical composition comprising (a) an active agent and (b) an oleaginous base comprising a skin penetration enhancer in an amount of less than about 20% w/w based on the total weight of the composition.

In some aspects of the present application, the skin penetration enhancer is present in an amount of less than about 20% w/w or an amount of less than about 19%, or in an amount of less than about 18% or in an amount of less than about 17% or in an amount of less than about 16% or in an amount of less than about 15% w/w or in an amount of less than about 14% w/w or in an amount of less than about 13% w/w or in an amount of less than about 12% w/w or in an amount of less than about 11% w/w or in an amount of less than about 10% w/w or in an amount of less than about 9% w/w or in an amount of less than about 8% w/w or in an amount of less than about 7% w/w or in an amount of less than about 6% w/w or in an amount of less than about 5% w/w or in an amount of less than about 4% w/w or in an amount of less than about 3% w/w or in an amount of less than about 2% w/w or in an amount of less than about 1% w/w based on the total weight of the composition.

In an aspect of the present application, the topical composition comprises (a) an active agent and (b) an oleaginous base comprising (i) one or more stiffening agent(s), (ii) a skin penetration enhancer and (iii) an oleaginous vehicle having melting point more than about 35° C.; wherein the skin penetration enhancer is liquid at room temperature.

In some aspects of the present application, the skin penetration enhancer is present in an amount of less than about 20% w/w based on the total weight of the composition.

In some aspects of the present application, the skin penetration enhancer is a water-immiscible substance and acts as an emollient in the composition.

An aspect of the present application relates to a topical composition comprising (a) an active agent and (b) an oleaginous base comprising (i) one or more stiffening agent(s), (ii) a skin penetration enhancer and (iii) an oleaginous vehicle having melting point more than about 35° C.; wherein the weight ratio between the skin penetration enhancer and the oleaginous vehicle is in the range of from about 1:5 to about 1:9.5. In some embodiments, the weight ratio between the skin penetration enhancer and the oleaginous vehicle is about 2:7, 1:5, 2:11, 1:6, 1:13, 1:7, 2:15, 1:8, 2:17, 1:9, 2:19, 1:10, 1:11, 1:12, 1:13, 1:14, 1:15, 1:16, 1:17, and 1:18.

The present application relates to an oleaginous base comprising (a) an active agent and (b) an oleaginous vehicle; wherein the oleaginous base further comprises one or more agent selected from group consisting of preservative, polymer, stiffening agent, water, water-miscible substance, water-immiscible substance, solvent, skin penetration enhancer, surfactant, emulsifying agent, antioxidant, and combinations thereof.

An aspect of the present application relates to a topical composition comprising an active agent and an oleaginous base; wherein the oleaginous base comprises (i) a stiffening agent, (ii) a skin penetration enhancer and (iii) an oleaginous vehicle.

In some aspects of the present application, the stiffening agent(s) can interchangeably be used for bodifying agent or thickening agent.

In an aspect of the present application, the stiffening agent is selected from white wax, microcrystalline wax, emulsifying wax, colloidal silicon dioxide, and the like.

In an aspect of the present application, the stiffening agent is present in an amount of 0% or in an amount of about 0% or in an amount of about 1%, or in an amount of about 2%, or in an amount of about 3%, or in an amount of about 4%, or in an amount of about 5%, or in an amount of about 6%, or in an amount of about 7%, or in an amount of about 8%, or in an amount of about 9%, or in an amount of about 10%, or in an amount of about 11%, or in an amount of about 12%, or in an amount of about 13%, or in an amount of about 14%, or in an amount of about 15%, or in an amount of about 16%, or in an amount of about 17%, or in an amount of about 18%, or in an amount of about 19%, or in an amount of about 20%, or in an amount of about 21%, or in an amount of about 22%, or in an amount of about 23%, or in an amount of about 24%, or in an amount of about 25%, or in an amount of about 26%, or in an amount of about 27%, or in an amount of about 28%, or in an amount of about 29%, or in an amount of about 30%, or in an amount of about 31%, or in an amount of about 32%, or in an amount of about 33%, or in an amount of about 34%, or in an amount of about 35%, or in an amount of about 36%, or in an amount of about 37%, or in an amount of about 38%, or in an amount of about 39%, or in an amount of about 40%, or in an amount of about 41%, or in an amount of about 42%, or in an amount of about 43%, or in an amount of about 44%, or in an amount of about 45%, or in an amount of about 46%, or in an amount of about 47%, or in an amount of about 48%, or in an amount of about 49%, or in an amount of about 50% based on the total weight of the composition.

In some aspects of the present application, the topical composition is substantially free of the polymeric substance.

In some aspects of the present application, the topical composition is free of a polymeric substance such as cellulose, carbomers, and the like.

In an aspect of the present application, the stiffening agent aids in preventing the phase separation or sedimentation or syneresis of the composition for a period of at least about three months.

In some aspects of the present application, the oleaginous base comprises one or more water-immiscible substance(s) which will act as a stiffening agent in the composition.

In some aspects of the present application, the stiffening agent(s) is selected from one or more substances from the group of anionic emulsifying wax, beeswax, carnauba wax, cetyl esters wax, non-ionic emulsifying wax, white wax, and yellow wax.

In some aspects of the present application, the stiffening agent is white wax and is present in the range of from about 1% w/w to about 45% w/w, or about 1% w/w to about 40% w/w, or about 1% w/w to about 35% w/w, or about 1% w/w to about 30% w/w, or about 1% w/w to about 25% w/w, or about 1% w/w to about 20% w/w, or about 1% w/w to about 15% w/w, or about 1% w/w to about 10% w/w, or about 1% w/w to about 5% w/w based on the total weight of the composition

In some aspects of the present application, the stiffening agent is microcrystalline wax and is present in the range of from about 1% w/w to about 45% w/w, or about 1% w/w to about 40% w/w, or about 1% w/w to about 35% w/w, or about 1% w/w to about 30% w/w, or about 1% w/w to about 25% w/w, or about 1% w/w to about 20% w/w, or about 1% w/w to about 15% w/w, or about 1% w/w to about 10% w/w, or about 1% w/w to about 5% w/w based on the total weight of the composition.

In some aspects of the present application, the stiffening agent is emulsifying wax and is present in the range of from about 1% w/w to about 45% w/w, or about 1% w/w to about 40% w/w, or about 1% w/w to about 35% w/w, or about 1% w/w to about 30% w/w, or about 1% w/w to about 25% w/w, or about 1% w/w to about 20% w/w, or about 1% w/w to about 15% w/w, or about 1% w/w to about 10% w/w, or about 1% w/w to about 5% w/w based on the total weight of the composition.

In some aspects of the present application, the stiffening agent is cetyl esters wax and is present in the range of from about 1% w/w to about 45% w/w, or about 1% w/w to about 40% w/w, or about 1% w/w to about 35% w/w, or about 1% w/w to about 30% w/w, or about 1% w/w to about 25% w/w, or about 1% w/w to about 20% w/w, or about 1% w/w to about 15% w/w, or about 1% w/w to about 10% w/w, or about 1% w/w to about 5% w/w based on the total weight of the composition.

In some aspects of the present application, the stiffening agent is yellow wax and is present in the range of from about 1% w/w to about 45% w/w, or about 1% w/w to about 40% w/w, or about 1% w/w to about 35% w/w, or about 1% w/w to about 30% w/w, or about 1% w/w to about 25% w/w, or about 1% w/w to about 20% w/w, or about 1% w/w to about 15% w/w, or about 1% w/w to about 10% w/w, or about 1% w/w to about 5% w/w based on the total weight of the composition.

In some aspects of the present application, the stiffening agent is beeswax and is present in the range of from about 1% w/w to about 45% w/w, or about 1% w/w to about 40% w/w, or about 1% w/w to about 35% w/w, or about 1% w/w to about 30% w/w, or about 1% w/w to about 25% w/w, or about 1% w/w to about 20% w/w, or about 1% w/w to about 15% w/w, or about 1% w/w to about 10% w/w, or about 1% w/w to about 5% w/w based on the total weight of the composition.

In some aspects of the present application, the stiffening agent has a melting point more than about 35° C.

In some aspects of the present application, the stiffening agent has a melting point from about 38° C. to about 40° C.

In some aspects of the present application, the stiffening agent has a melting point from more than about 40° C.

In some aspects of the present application, the stiffening agent has a melting point from more than about 50° C.

In some aspects of the present application, the stiffening agent has a melting point from more than about 60° C.

In some aspects of the present application, the stiffening agent has a melting point from about 40° C. to about 120° C.

In some aspects of the present application, the stiffening agent has a melting point of about 35° C. or about 36° C. or about 37° C. or about 38° C. or about 39° C. or about 40° C. or about 41° C. or about 42° C. or about 43° C. or about 44° C. or about 45° C. or about 46° C. or about 47° C. or about 48° C. or about 49° C. or about 50° C. or about 51° C. or about 52° C. or about 53° C. or about 54° C. or about 55° C. or about 56° C. or about 57° C. or about 58° C. or about 59° C. or about 60° C. or about 61° C. or about 62° C. or about 63° C. or about 64° C. or about 65° C. or about 66° C. or about 67° C. or about 68° C. or about 69° C. or about 70° C. or about 71° C. or about 72° C. or about 73° C. or about 74° C. or about 75° C. or about 76° C. or about 77° C. or about 78° C. or about 79° C. or about 80° C. or about 81° C. or about 82° C. or about 83° C. or about 84° C. or about 85° C. or about 86° C. or about 87° C. or about 88° C. or about 89° C. or about 90° C. or about 91° C. or about 92° C. or about 93° C. or about 94° C. or about 95° C. or about 96° C. or about 97° C. or about 98° C. or about 99° C. or about 100° C. or about 101° C. or about 102° C. or about 103° C. or about 104° C. or about 105° C. or about 106° C. or about 107° C. or about 108° C. or about 109° C. or about 110° C. or about 111° C. or about 112° C. or about 113° C. or about 114° C. or about 115° C. or about 116° C. or about 117° C. or about 118° C. or about 119° C. or about 120° C.

In some aspects of the present application, the stiffening agent comprises at least two stiffening agents selected from white wax, microcrystalline wax, emulsifying wax, cetyl esters wax, yellow wax, beeswax, and the like.

In some aspects of the present application, the stiffening agent comprises at least two stiffening agents in the weight ratio of from about 1:1 to about 3:1. In some embodiments, the weight ratio between the two stiffening agents is about 1:1, 3:2, 2:1, 5:2, 3:1, or 7:2.

An aspect of the present application relates to a topical composition comprising (a) an active agent and (b) an oleaginous base comprising: (i) at least two stiffening agents and (ii) an oleaginous vehicle; wherein the stiffening agents selected from white wax, microcrystalline wax, emulsifying wax, cetyl esters wax, yellow wax, beeswax and, any combination thereof; wherein the weight ratio between the two stiffening agent(s) is in the range of from about 1:1 to about 3:1. In some embodiments, the weight ratio between the two stiffening agents is about 1:1, 3:2, 2:1, 5:2, 3:1, or 7:2.

In some aspects of the present application, the oleaginous vehicle is one or more excipient(s) selected from mineral oil, soft paraffin, hard paraffin, petrolatum, mixture of mineral oil and lanolin alcohols, coconut oil, almond oil, lanolin, mixture of petrolatum and lanolin alcohols, fatty alcohols, vegetable oils, and combinations thereof.

In some aspects of the present application, the oleaginous vehicle is one or more water-immiscible substance(s) that are present in the composition in an amount of at least about 50% w/w based on the total weight of the composition.

In some aspects of the present application, the oleaginous vehicle is one or more excipient(s) selected from mineral oil, soft paraffin, hard paraffin, petrolatum, mixture of mineral oil and lanolin alcohols, coconut oil, almond oil, lanolin, mixture of petrolatum and lanolin alcohols, fatty alcohols, vegetable oils, and combinations thereof.

In some aspects of the present application, the oleaginous vehicle has a melting point of about 35° C. or about 36° C. or about 37° C. or about 38° C. or about 39° C. or about 40° C. or about 41° C. or about 42° C. or about 43° C. or about 44° C. or about 45° C. or about 46° C. or about 47° C. or about 48° C. or about 49° C. or about 50° C. or about 51° C. or about 52° C. or about 53° C. or about 54° C. or about 55° C. or about 56° C. or about 57° C. or about 58° C. or about 59° C. or about 60° C. or about 61° C. or about 62° C. or about 63° C. or about 64° C. or about 65° C. or about 66° C. or about 67° C. or about 68° C. or about 69° C. or about 70° C. or about 71° C. or about 72° C. or about 73° C. or about 74° C. or about 75° C. or about 76° C. or about 77° C. or about 78° C. or about 79° C. or about 80° C. or about 81° C. or about 82° C. or about 83° C. or about 84° C. or about 85° C. or about 86° C. or about 87° C. or about 88° C. or about 89° C. or about 90° C. or about 91° C. or about 92° C. or about 93° C. or about 94° C. or about 95° C. or about 96° C. or about 97° C. or about 98° C. or about 99° C. or about 100° C. or about 101° C. or about 102° C. or about 103° C. or about 104° C. or about 105° C. or about 106° C. or about 107° C. or about 108° C. or about 109° C. or about 110° C. or about 111° C. or about 112° C. or about 113° C. or about 114° C. or about 115° C. or about 116° C. or about 117° C. or about 118° C. or about 119° C. or about 120° C.

In some aspects of the present application, the oleaginous vehicle has a melting point from more than about 35° C.

In some aspects of the present application, the oleaginous vehicle has a melting point from about 38° C. to about 40° C.

In some aspects of the present application, the oleaginous vehicle has a melting point from more than about 40° C.

In some aspects of the present application, the oleaginous vehicle has a melting point from more than about 50° C.

In some aspects of the present application, the oleaginous vehicle has a melting point from more than about 60° C.

In some aspects of the present application, the oleaginous vehicle has a melting point from about 40° C. to about 120° C. In some aspects of the present application, the oleaginous vehicle comprises in an amount of at least about 40% w/w of the total weight of the oleaginous base.

In some aspects of the present application, the oleaginous vehicle comprises in an amount of at least about 50% w/w of the total weight of the oleaginous base.

In some aspects of the present application, the oleaginous vehicle comprises in an amount of at least about 60% w/w of the total weight of the oleaginous base.

In some aspects of the present application, the oleaginous vehicle comprises in an amount of at least about 70% w/w of the total weight of the oleaginous base.

In an aspect of the present application, the oleaginous base comprises the stiffening agent(s) and the oleaginous vehicle in a weight ratio between about 1:50 and about 1:1, and the oleaginous base optionally comprises one or more pharmaceutically acceptable excipient(s) such as preservative(s), antioxidant, and the like.

In some aspects of the present application, the oleaginous base comprises the stiffening agent(s) and the oleaginous vehicle in a weight ratio of about 1:45.

In some aspects of the present application, the oleaginous base comprises the stiffening agent(s) and the oleaginous vehicle in a weight ratio of about 1:40.

In some aspects of the present application, the oleaginous base comprises the stiffening agent(s) and the oleaginous vehicle in a weight ratio of about 1:35.

In some aspects of the present application, the oleaginous base comprises the stiffening agent(s) and the oleaginous vehicle in a weight ratio of about 1:30.

In some aspects of the present application, the oleaginous base comprises the stiffening agent(s) and the oleaginous vehicle in a weight ratio of about 1:25.

In some aspects of the present application, the oleaginous base comprises the stiffening agent(s) and the oleaginous vehicle in a weight ratio of about 1:20.

In some aspects of the present application, the oleaginous base comprises the stiffening agent(s) and the oleaginous vehicle in a weight ratio of about 1:15.

In some aspects of the present application, the oleaginous base comprises the stiffening agent(s) and the oleaginous vehicle in a weight ratio of about 1:10.

In some aspects of the present application, the oleaginous base comprises the stiffening agent(s) and the oleaginous vehicle in a weight ratio of about 1:7.

In some aspects of the present application, the oleaginous base comprises the stiffening agent(s) and the oleaginous vehicle in a weight ratio of about 1:5.

In some aspects of the present application, the oleaginous base comprises the stiffening agent(s) and the oleaginous vehicle in a weight ratio of about 1:1.

An aspect of the present application relates to a topical composition comprising (a) an active agent, (b) one or more stiffening agent(s), (c) a skin penetration enhancer, (d) an oleaginous vehicle, and (e) a pharmaceutically acceptable excipient(s); wherein the composition comprises the stiffening agent, the skin penetration enhancer, and the oleaginous vehicle in a weight ratio from about 3:2:14 to about 2:1:17.

An aspect of the present application relates to an oleaginous base comprising (a) an active agent (b) one or more stiffening agent(s), (c) a skin penetration enhancer (d) an oleaginous vehicle having a melting point more than about 35° C. and (e) one or more pharmaceutically acceptable excipient(s); wherein the composition comprises the stiffening agent(s), the skin penetration enhancer, and the oleaginous vehicle in the weight ratio from about 3:2:14 to about 2:1:17.

In some aspects of the present application, the weight ratio between the stiffening agent(s), the skin penetration enhancer, and the oleaginous vehicle is essential for the physical stability of the composition and is selected from about 3:3:13, about 3:2:14, about 3:2:15, about 2:2:15, about 2:2:16, about 3:1:15, about 3:1:14, and about 2:1:16. In some embodiments, the weight ratio between the stiffening agent(s), the skin penetration enhancer, and the oleaginous vehicle is about 2:1:13, 2:1:14, 2:1:15, 2:1:16, 2:1:17, 2:1:18, 3:2:13, 3:2:14, 3:2:15, 3:2:16, 3:2:17, or 3:2:18. In some aspects of the present application, the oleaginous base optionally comprises a surfactant, a water-miscible substance(s), a polymer, one or more preservative(s), a solvent, a water-immiscible substance, a pH modifying agent, water, and combinations thereof.

In an aspect of the present application, the topical composition comprises an oleaginous base that releases an active agent in a controlled manner.

In an aspect of the present application, the topical composition comprises an oleaginous base comprising an oleaginous vehicle in an amount of at least about 60% w/w based on the total weight of the composition.

In an aspect, the present application relates to a topical composition comprising an active agent and an oleaginous base; wherein the oleaginous base comprises one or more water-miscible substance(s) in an amount of less than about 10% w/w based on the total weight of the composition.

In an aspect, the present application relates to a topical composition comprising an active agent and an oleaginous base; wherein the composition comprises one or more water-immiscible substance(s) in an amount of at least 90% w/w based on the total weight of the composition.

In some aspects of the present application, the weight ratio between a water-immiscible substance(s) to a water-miscible substance(s) is in the range from about 9:1 to about 9:0.1.

In an aspect, the present application relates to a topical composition comprising (a) an active agent (b) one or more stiffening agent(s), (c) a skin penetration enhancer, (d) an oleaginous vehicle having a melting point more than about 35° C., and (e) one or more pharmaceutically acceptable excipient(s); wherein the composition releases the active agent in a controlled manner.

In an aspect of the present application, the topical composition comprises (a) an active agent and (b) an oleaginous base comprising a skin penetration enhancer; wherein the composition releases the active agent less than about 0.9 milligram/cm2/hour when measured using an in vitro release testing system utilizing Franz diffusion cell and the following conditions: receptor media comprising 60% of ethanol, 40% of water, cellulose nitrate as membrane, 400-700 rpm, infinite dosing, and temperature in the range of 30° C.±10° C.

In some aspects of the present application, the topical composition releases the active agent less than about 0.8 milligram/cm²/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In some aspects of the present application, the topical composition releases the active agent less than about 0.7 milligram/cm²/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In some aspects of the present application, the topical composition releases the active agent less than about 0.6 milligram/cm²/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In some aspects of the present application, the topical composition releases the active agent less than about 0.5 milligram/cm²/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In some aspects of the present application, the topical composition releases the active agent less than about 0.4 milligram/cm²/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In some aspects of the present application, the topical composition releases the active agent less than about 0.3 milligram/cm²/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In some aspects of the present application, the topical composition releases the active agent less than about 0.2 milligram/cm²/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In some aspects of the present application, the topical composition releases the active agent less than about 0.1 milligram/cm²/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In some aspects of the present application, the topical composition releases the active agent in the range of from about 0.001 milligram/cm²/hour to about 0.1 milligram/cm²/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In an aspect of the present application, the topical composition comprises (a) an active agent and (b) an oleaginous base comprising a skin penetration enhancer; wherein the composition releases the active agent in the range of from about 0.001 milligram/cm²/hour to about 0.1 milligram/cm²/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.; and the oleaginous base is present in an amount of at least about 60% w/w based on the total weight of the composition.

In some aspects of the present application, the active agent is selected from a corticosteroid, a topical calcineurin inhibitor, antibiotic, anti-histamine, NSAID, COX-II inhibitor, antifungal, vitamin D or analogs, phosphodiesterase 4 (PDE4) inhibitor, 5-lipoxygenase inhibitor, retinoid compound, immunomodulator, and the like.

In some aspects of the present application, the active agent is selected from betamethasone, clobetasol, dexamethasone, mometasone, halobetasol, tretinoin, tazarotene, adapalene, tacrolimus, pimecrolimus doxepin, zileuton, cetirizine, diclofenac, ibuprofen, crisaborole, erythromycin, doxycycline, minocycline, celecoxib, mupirocin, miconazole, calcitriol, calcipotriene, retapamulin, chlorpheniramine and its pharmaceutically acceptable salts, prodrugs, esters, solvates, polymorphs thereof.

In some aspects of the present application, the active agent is tacrolimus, zileuton, crisaborole, doxepin, and its pharmaceutically acceptable salts, prodrugs, esters, solvates, polymorphs thereof.

In an aspect of the present application, the topical composition comprises (a) zileuton and (b) an oleaginous base comprising a skin penetration enhancer; wherein the composition releases the active agent less than about 0.05 milligram/cm2/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In an aspect of the present application, the topical composition comprises (a) zileuton and (b) an oleaginous base comprising a skin penetration enhancer; wherein the composition releases the active agent less than about 0.04 milligram/cm2/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In an aspect of the present application, the topical composition comprises (a) zileuton and (b) an oleaginous base comprising a skin penetration enhancer; wherein the composition releases the active agent less than about 0.03 milligram/cm2/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In an aspect of the present application, the topical composition comprises (a) zileuton and (b) an oleaginous base comprising a skin penetration enhancer; wherein the composition releases the active agent less than about 0.02 milligram/cm2/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In an aspect of the present application, the topical composition comprises (a) zileuton and (b) an oleaginous base comprising a skin penetration enhancer; wherein the composition releases the active agent less than about 0.01 milligram/cm2/hour when measured using an in vitro release testing system utilizing Franz diffusion cell under the following conditions: receptor media comprising 60% of ethanol, 40% of water, cellulose nitrate as membrane, 400-700 rpm, infinite dosing, and temperature in the range of 30° C.±10° C.

In an aspect of the present application, the topical composition comprises (a) zileuton and (b) an oleaginous base comprising a skin penetration enhancer; wherein the composition releases the active agent at least about 0.01 milligram/cm2/hour and not more than about 0.05 milligram/cm2/two hour when measured using an in vitro release testing system utilizing Franz diffusion cell and the following conditions: receptor media comprising 60% of ethanol, 40% of water, cellulose nitrate as membrane, 400-700 rpm, infinite dosing, and temperature in the range of 30° C.±10° C.

In an aspect of the present application, the topical composition comprises (a) zileuton and (b) an oleaginous base comprising a skin penetration enhancer; wherein the composition releases the active agent at least about 0.02 milligram/cm2/three hour and not more than about 0.06 milligram/cm2/three hour when measured using an in vitro release testing system utilizing Franz diffusion cell and the following conditions: receptor media comprising 60% of ethanol, 40% of water, cellulose nitrate as membrane, 400-700 rpm, infinite dosing, and temperature in the range of 30° C.±10° C.

In an aspect of the present application, the topical composition comprises (a) zileuton and (b) an oleaginous base comprising a skin penetration enhancer; wherein the composition releases the active agent in the range of from about 0.001 milligram/cm²/hour to about 0.1 milligram/cm²/hour when measured using an in vitro release testing system utilizing Franz diffusion cell and the following conditions: receptor media comprising 60% of ethanol, 40% of water, cellulose nitrate as membrane, 400-700 rpm, infinite dosing, temperature in the range of 30° C.±10° C., and the oleaginous base is present in an amount of at least about 60% w/w based on the total weight of the composition.

In an aspect of the present application, the topical composition comprises an active agent in non-solubilized form or partially solubilized form.

In an aspect of the present application, the topical composition comprises an active agent having a D90 particle size of less than about 50 microns.

In an aspect of the present application, the topical composition comprises an active agent having a D90 particle size of less than about 40 microns.

In an aspect of the present application, the topical composition comprises an active agent having a D90 particle size of less than about 30 microns.

In an aspect of the present application, the topical composition comprises an active agent having a D90 particle size of less than about 20 microns.

In an aspect of the present application, the topical composition comprises an active agent having a D90 particle size of less than about 10 microns.

In an aspect of the present application, the topical composition comprises an active agent having a D90 particle size of less than about 5 microns.

In an aspect of the present application, the topical composition comprises an active agent having a D90 particle size of less than about 3 microns.

In an aspect of the present application, the oleaginous composition is a monophasic system or biphasic system.

In an aspect of the present application, the topical composition is substantially free of water or water-miscible substance(s).

In an aspect of the present application, the topical composition comprises water or water-miscible substance(s) in an amount of less than about 10% w/w or in an amount of less than about 9% w/w or in an amount of less than about 8% w/w or in an amount of less than about 7% w/w, or in an amount of less than about 6% w/w or in an amount of less than about 5% w/w or in an amount of less than about 4% w/w or in an amount of less than about 3% w/w or in an amount of less than about 2% w/w or in an amount of less than about 1% w/w or in an amount of less than about 0.5% w/w or in an amount of less than about 0% w/w based on the total weight of the composition.

In an aspect of the present application, the topical composition is substantially free of the water-miscible or hydrophilic substance. The water-miscible or hydrophilic substance includes, but are not limited, diethylene glycol monoethyl ether, propylene glycol, polypropylene glycol, polyethylene glycol, ethanol, isopropyl alcohol, glycerine, and the like.

In an aspect of the present application, the topical composition is substantially free of hydrophilic solvent(s) such as diethylene glycol monoethyl ether, ethanol, isopropyl alcohol, propylene glycol, polyethylene glycol, and the like.

In an aspect of the present application, the topical composition comprises one or more skin penetration enhancer(s). The skin penetration enhancer is selected from the water-miscible substance(s), water-immiscible substance, and combinations thereof.

In an aspect of the present application, the topical composition is substantially free of propylene glycol.

In an aspect of the present application, the topical composition comprises propylene glycol in an amount of less than about 10% w/w or in an amount of less than about 9% w/w or in an amount of less than about 8% w/w or in an amount of less than about 7% w/w, or in an amount of less than about 6% w/w or in an amount of less than about 5% w/w or in an amount of less than about 4% w/w or in an amount of less than about 3% w/w or in an amount of less than about 2% w/w or in an amount of less than about 1% w/w or in an amount of less than about 0% w/w based on the total weight of the composition.

In an aspect of the present application, the topical composition is free of propylene glycol.

In an aspect of the present application, the topical composition comprises water-immiscible substance.

In some aspects of the present application, the topical composition comprises fatty acid esters as a skin penetration enhancer.

In an aspect of the present application, the topical composition comprises isopropyl myristate.

In some aspects of the present application, the skin penetration enhancer is isopropyl myristate, and is present in an amount of less than about 20% w/w based on the total weight of the composition or in an amount of less than about 15% w/w or in amount of less than about 10% w/w or amount of less than about 5% w/w based on the total weight of the composition.

In some aspects of the present application, the skin penetration enhancer is isopropyl myristate and is present in an amount of about 10% w/w based on the total weight of the composition.

In some aspects of the present application, the skin penetration enhancer is isopropyl palmitate, and is present in an amount of less than about 20% w/w or in an amount of less than about 15% w/w or in an amount of less than about 10% w/w or in an amount of less than about 5% w/w or about 10% w/w based on the total weight of the composition

In some aspects of the present application, the skin penetration enhancer is diisopropyl adipate, and is present in an amount of less than about 20% w/w or in an amount of less than about 15% w/w or in an amount of less than about 10% w/w or in an amount of less than about 5% w/w or in an amount of about 10% w/w based on the total weight of the composition.

In some aspects of the present application, the skin penetration enhancer is dibutyl sebacate, and is present in an amount of less than about 20% w/w or in an amount of less than about 15% w/w or in an amount of less than about 10% w/w or in an amount of less than about 5% w/w or in an amount of about 10% w/w based on the total weight of the composition.

In some aspects of the present application, the skin penetration enhancer is myristyl alcohol, and is present in an amount of less than about 20% w/w or in an amount of less than about 15% w/w or in an amount of less than about 10% w/w or in an amount of less than about 5% w/w or in an amount of about 10% w/w based on the total weight of the composition.

In some aspects of the present application, the skin penetration enhancer is octyl dodecanol, and is present in an amount of less than about 20% w/w or in an amount of less than about 15% w/w or in an amount of less than about 10% w/w or in an amount of less than about 5% w/w or in an amount of about 10% w/w based on the total weight of the composition.

In some aspects of the present application, the skin penetration enhancer is stearic acid, and is present in an amount of less than about 20% w/w or in an amount of less than about 15% w/w or in an amount of less than about 10% w/w or in an amount of less than about 5% w/w or in an amount of about 10% w/w based on the total weight of the composition.

In some aspects of the present application, the skin penetration enhancer is oleyl alcohol, and is present in an amount of less than about 20% w/w or in an amount of less than about 15% w/w or in an amount of less than about 10% w/w or in an amount of less than about 5% w/w or in an amount of about 10% w/w based on the total weight of the composition.

In some aspects of the present application, the skin penetration enhancer is stearyl alcohol, and is present in an amount of less than about 20% w/w or in an amount of less than about 15% w/w or in an amount of less than about 10% w/w or in an amount of less than about 5% w/w or in an amount of about 10% w/w based on the total weight of the composition.

In some aspects of the present application, the skin penetration enhancer is oleic acid, and is present in an amount of less than about 20% w/w or in an amount of less than about 15% w/w or in an amount of less than about 10% w/w or in an amount of less than about 5% w/w or in an amount of about 10% w/w based on the total weight of the composition.

In some aspects of the present application, the skin penetration enhancer is substantially free of hydrophilic solvent(s).

In some aspects of the present application, the composition is substantially free of skin penetration enhancer.

In some aspects of the present application, the composition is substantially free of glycol(s).

In some aspects of the present application, the composition is substantially free of propylene glycol.

In some aspects of the present application, the composition is substantially free of polyethylene glycol.

In some aspects of the present application, the composition comprises glycol(s) lesser than the active agent solubilizing level.

In some aspects of the present application, the composition is substantially free of preservative(s).

In some aspects of the present application, the composition is substantially free of surfactant(s) and/or amphiphilic substance(s).

In some aspects of the present application, the composition is substantially free of antioxidant(s).

In some aspects of the present application, the composition is non-foaming or non-foamable composition.

In some aspects of the present application, the composition of the present application is occlusive and forms a thin oily film at the application site.

In some aspects of the present application, the composition is substantially anhydrous.

In some aspects of the present application, the composition is propellant-free, i.e. not delivered using propellant(s).

In some aspects of the present application, the composition is substantially free of hyaluronic acid.

In some aspects of the present application, the active agent is in non-solubilized form.

In some aspects of the present application, the active agent is in a partially solubilized form.

In some aspects of the present application, the active agent is in solubilized form.

In some aspects of the present application, the active agent is a non-steroidal active agent.

In some aspects of the present application, the active agent is a non-steroidal active agent and is selected from the group consisting of tretinoin, tazarotene, adapalene, tacrolimus, pimecrolimus doxepin, zileuton, cetirizine, diclofenac, ibuprofen, crisaborole, erythromycin, doxycycline, minocycline, celecoxib, mupirocin, miconazole, calcitriol, calcipotriene, retapamulin, chlorpheniramine and its pharmaceutically acceptable salts, prodrugs, esters, solvates, polymorphs thereof and combinations thereof.

An aspect of the present application relates to a topical composition comprising (a) zileuton, (b) one or more stiffening agent(s), (c) a skin penetration enhancer, (d) an oleaginous vehicle, and (e) one or more pharmaceutically acceptable excipient(s).

In an aspect of the present application, the topical composition comprises zileuton as an active agent.

An aspect of the present application relates to a topical composition comprising zileuton and an oleaginous base; wherein the oleaginous base is substantially free of water.

An aspect of the present application relates to a topical composition comprising zileuton and an oleaginous base; wherein the oleaginous base comprises an oleaginous vehicle in an amount of at least about 60% w/w based on the total weight of the composition.

An aspect of the present application relates to a topical composition comprising zileuton and an oleaginous base; wherein the oleaginous base comprises a skin penetration enhancer in an amount of less than about 20% w/w based on the total weight of the composition.

An aspect of the present application relates to a topical composition comprising zileuton and an oleaginous base; wherein the oleaginous base comprises a skin penetration enhancer in an amount of less than about 15% w/w based on the total weight of the composition.

An aspect of the present application relates to a topical composition comprising zileuton and an oleaginous base; wherein the oleaginous base comprises a skin penetration enhancer in an amount of less than about 10% w/w based on the total weight of the composition.

An aspect of the present application relates to a topical composition comprising zileuton and an oleaginous base; wherein the oleaginous base comprises a skin penetration enhancer in an amount of about 10% w/w based on the total weight of the composition.

An aspect of the present application relates to a topical composition comprising zileuton and an oleaginous base; wherein the oleaginous base comprises a skin penetration enhancer and an oleaginous vehicle in an amount of at least about 60% w/w based on the total weight of the composition wherein the skin penetration enhancer is in the form of a liquid at room temperature.

An aspect of the present application relates to a topical composition comprising zileuton and an oleaginous base; wherein the oleaginous base comprises (i) at least two stiffening agents and (ii) an oleaginous vehicle; wherein the stiffening agents selected from white wax, microcrystalline wax, emulsifying wax, cetyl esters wax, yellow wax, beeswax and any combination thereof, wherein the weight ratio between the two stiffening agents is in the range of from about 1:1 to about 3:1. In some embodiments, the weight ratio between the stiffening agent(s) and the skin penetration enhancer is about 3:1, 2:1, 3:2, 6:5, or 1:1.

An aspect of the present application relates to a topical composition comprising zileuton and an oleaginous base; wherein the oleaginous base comprises (i) one or more stiffening agent(s), (ii) a skin penetration enhancer and (iii) an oleaginous vehicle having melting point more than about 35° C.; wherein the weight ratio between the skin penetration enhancer and the oleaginous vehicle is in the range of from about 1:5 to about 1:9.5. In some embodiments, the weight ratio between the skin penetration enhancer and the oleaginous vehicle is about 2:7, 1:5, 2:11, 1:6, 1:13, 1:7, 2:15, 1:8, 2:17, 1:9, 2:19, 1:10, 1:11, 1:12, 1:13, 1:14, 1:15, 1:16, 1:17, and 1:18.

An aspect of the present application relates to a topical composition comprising zileuton and an oleaginous base; wherein the oleaginous base comprises (i) one or more stiffening agent(s), (ii) a skin penetration enhancer and (iii) an oleaginous vehicle; wherein the weight ratio between the stiffening agent(s) and the oleaginous vehicle is in the range of from about 1:45 to about 1:1. In some embodiments, the weight ratio between the stiffening agent(s) and the oleaginous vehicle is about 1:50, 1:45, 1:40, 1:35, 1:30, 1:25, 1:20, 1:15, 1:10, 1:5, or 1:1. In some embodiments, the weight ratio between the stiffening agent(s) and the skin penetration enhancer is in the range of from about 3:2 to about 1:1. In some embodiments, the weight ratio between the stiffening agent(s) and the skin penetration enhancer is about 3:1, 2:1, 3:2, 6:5, or 1:1.

An aspect of the present application relates to a topical composition comprising (a) zileuton, (b) one or more stiffening agent(s), (c) a skin penetration enhancer, (d) an oleaginous vehicle having a melting point more than about 35° C., and (e) one or more pharmaceutically acceptable excipient(s); wherein the composition comprises a stiffening agent, a skin penetration enhancer, and an oleaginous vehicle in the weight ratio from about 3:2:14 to about 2:1:17. In some embodiments, the weight ratio between the stiffening agent(s), the skin penetration enhancer, and the oleaginous vehicle is about 2:1:13, 2:1:14, 2:1:15, 2:1:16, 2:1:17, 2:1:18, 3:2:13, 3:2:14, 3:2:15, 3:2:16, 3:2:17, or 3:2:18. In some embodiments, the weight ratio between the stiffening agent(s), the skin penetration enhancer, and the oleaginous vehicle is about 2:1:13, 2:1:14, 2:1:15, 2:1:16, 2:1:17, 2:1:18, 3:2:13, 3:2:14, 3:2:15, 3:2:16, 3:2:17, or 3:2:18.

In an aspect, the present application relates to a topical composition comprising (a) zileuton and (b) an oleaginous base; wherein the composition has an oleaginous base in an amount of at least about 60% w/w based on the total weight of the composition and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

Some aspects of the present application relates to a topical composition comprising (a) zileuton and (b) an oleaginous base; wherein the composition has an oleaginous base in an amount of at least about 70% w/w based on the total weight of the composition and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

In an aspect of the present application, the topical composition provides release of zileuton in a controlled manner.

In an aspect of the present application, the topical composition additionally comprises a water-miscible substance(s); wherein the weight ratio between the water-immiscible substance(s) and the water-miscible substance(s) is in the range from about 9:1 to about 9:0.1. In some embodiments, the weight ratio between the water-immiscible substance(s) and water-miscible substance(s) is about 9:1.1, 9:1, 9:0.9, 9:0.8, 9:0.7, 9:0.6, 9:0.6, 9:0.4, 9:0.3, 9:0.2, or 9:0.1.

An aspect of the present application relates to a topical composition comprising zileuton; wherein zileuton is present in a non-solubilized form in the said composition.

An aspect of the present application relates to a topical composition comprising zileuton; wherein the D90 particle size of zileuton is less than about 50 microns.

An aspect of the present application relates to a topical composition comprising a) zileuton and b) one or more pharmaceutically acceptable excipient(s); wherein zileuton is present in a non-solubilized form in the said composition.

An aspect of the present application relates to a topical composition comprising a) zileuton and b) one or more pharmaceutically acceptable excipient(s); wherein the D90 particle size of zileuton is less than about 50 microns.

In an aspect of the present application, the D90 particle size of zileuton in the composition is less than about 40 microns, or less than about 30 microns, or less than about 20 microns, or less than about 10 microns, or less than about 5 microns, or about 3 microns.

An aspect of the present application relates to a topical composition comprising a) zileuton and b) one or more pharmaceutically acceptable excipient(s); wherein the composition is chemically and physically stable.

An aspect of the present application relates to a topical composition comprising a) zileuton and b) one or more pharmaceutically acceptable excipient(s); wherein the composition comprises less than about 30,000 ppm total related substance of zileuton and the maximum single known related substance will not exceed about 5000 ppm of total zileuton in the composition when stored under room temperature for at least about 3 months.

An aspect of the present application relates to a stable topical composition comprising a) zileuton and b) one or more pharmaceutically acceptable excipient(s); wherein the composition is chemically stable for at least about three months and provides total related substances of zileuton less than about 30,000 ppm, and a single known related substance of zileuton less than about 5000 ppm when stored at room temperature.

In some aspects of the present application, the D90 particle size of zileuton is from about 0.1 microns to about 10 microns.

In some aspects of the present application, the related substance of zileuton is selected from compound A (1-(1-(benzo[b]thiophen-2-yl)ethyl)urea), compound B (2-(Benzo[b]thien-2-oyl)benzo[b]thiophene), compound C (2-cetylbenzothiophene), compound D (primary amine derivative of zileuton-1-benzo[b]thien-2-ylethyamine), compound E (hydroxylamine derivative of zileuton-N-(1-benzo[b]thien-2-ylethyl)hydroxylamine), compound F (alcohol derivative of zileuton-1-benzo[b]thien-2-ylethyl alcohol), compound G (E-oxime derivative of zileuton-(E)-1-benzo[b]thien-2ylethanone oxime), and compound H (Z-oxime derivative of zileuton-(Z)-1-benzo[b]thien-2-ylethanone oxime).

An aspect of the present application relates to a topical composition comprising a) zileuton and b) an oleaginous base comprising one or more pharmaceutically acceptable excipient(s); wherein zileuton is present in a non-solubilized form in the said composition.

An aspect of the present application relates to a topical composition comprising a) zileuton and b) an oleaginous base comprising one or more pharmaceutically acceptable excipient(s); wherein D90 particle size of zileuton is less than about 50 microns.

An aspect of the present application relates to a topical composition comprising a) zileuton and b) an oleaginous base comprising one or more pharmaceutically acceptable excipient(s) wherein zileuton is present in a non-solubilized form in the said composition; wherein D90 particle size of zileuton is less than about 50 microns.

An aspect of the present application relates to a topical composition comprising a) zileuton and b) an oleaginous base comprising one or more pharmaceutically acceptable excipient(s); wherein the composition comprises less than about 30,000 ppm related substance of zileuton, and the maximum single known related substance will not exceed about 5000 ppm of total zileuton in the composition, when stored under room temperature for at least about 3 months.

An aspect of the present application relates to a topical composition comprising a) zileuton and b) an oleaginous base comprising one or more pharmaceutically acceptable excipient(s); wherein the composition is chemically stable for at least about 3 months and provides total related substances of zileuton less than about 30,000 ppm, and a single known related substance of zileuton less than about 5000 ppm when stored at room temperature.

An aspect of the present application relates to a topical composition comprising a) zileuton and b) an oleaginous base comprising in an amount of at least about 60% w/w of one or more water-immiscible substance(s) and in an amount of at least about 40% w/w of water or water-miscible substance based on the total weight of the oleaginous base.

An aspect of the present application relates to a topical composition comprising a) zileuton, b) an oleaginous base comprising one or more stiffening agent(s), and c) one or more pharmaceutically acceptable excipient(s); wherein D90 particle size of zileuton is less than about 50 microns.

An aspect of the present application relates to a topical composition comprising a) zileuton, b) an oleaginous base comprising stiffening agent(s) selected from the group consisting of anionic emulsifying wax, beeswax, carnauba wax, cetyl esters wax, non-ionic emulsifying wax, white wax, yellow wax, and mixtures thereof, and c) one or more pharmaceutically acceptable excipient(s); wherein the zileuton is present in a non-solubilized form and wherein the D90 particle size of zileuton is less than about 50 microns.

An aspect of the present application relates to a topical composition comprising a) zileuton, b) an oleaginous base comprising an oleaginous vehicle in an amount of at least about 40% w/w based on the total weight of an oleaginous base and one or more stiffening agent(s), and c) one or more pharmaceutically acceptable excipient(s).

An aspect of the present application relates to a topical composition comprising a) zileuton, b) an oleaginous base comprising an oleaginous vehicle in an amount of at least about 40% w/w based on the total weight of an oleaginous base and one or more stiffening agent(s), and c) one or more pharmaceutically acceptable excipient(s); wherein the zileuton is present in a non-solubilized form, and wherein the D90 particle size of zileuton is less than about 50 microns.

In some aspects of the present application, the oleaginous vehicle is white petrolatum and is present in an amount of at least about 40% w/w based on the total weight of the oleaginous base.

In some aspects of the present application, the oleaginous vehicle is white petrolatum and is present in an amount of at least about 50% w/w based on the total weight of the oleaginous base.

In some aspects of the present application, the oleaginous vehicle is white petrolatum and is present in an amount of at least about 60% w/w based on the total weight of the oleaginous base.

In some aspects of the present application, the oleaginous vehicle is white petrolatum and is present in an amount of at least about 70% w/w based on the total weight of the oleaginous base.

In some aspects of the present application, the oleaginous vehicle is paraffin and is present in an amount of at least about 40% w/w based on the total weight of the oleaginous base.

In some aspects of the present application, the oleaginous vehicle is lanolin alcohols and a mixture of lanolin alcohols with mineral oil, or petrolatum, and is present in an amount of at least about 40% w/w based on the total weight of the oleaginous base.

In some aspects of the present application, the oleaginous vehicle is lanolin alcohols and a mixture of lanolin alcohols with mineral oil, or petrolatum, and is present in an amount of at least about 50% w/w based on the total weight of the oleaginous base.

In some aspects of the present application, the oleaginous vehicle is lanolin alcohols and a mixture of lanolin alcohols with mineral oil, or petrolatum, and is present in an amount of at least about 60% w/w based on the total weight of the oleaginous base.

In some aspects of the present application, the oleaginous vehicle is lanolin alcohols and a mixture of lanolin alcohols with mineral oil, or petrolatum, and is present in an amount of at least about 70% w/w based on the total weight of the oleaginous base.

In an aspect of the present application, zileuton is present in a range of from about 1% to about 10% w/w based on the total weight of the composition or any skin tolerable concentration of zileuton may be used in the composition.

In a further aspect of the present application, zileuton is present in an amount of about 1%, or about 1.25%, or about 1.5%, or about 1.75%, or about 2%, or about 2.25%, or about 2.5%, or about 2.75%, or about 3%, or about 3.25%, or about 3.5%, or about 3.75%, or about 4%, or about 4.25%, or about 4.5%, or about 4.75%, or about 5%, or about 5.25%, or about 5.5%, or about 5.75%, or about 6%, or about 6.25%, or about 6.5%, or about 6.75%, or about 7%, or about 7.25%, or about 7.5%, or about 7.75%, or about 8%, or about 8.25%, or about 8.5%, or about 8.75%, or about 9%, or about 9.25%, or about 9.5%, or about 9.75%, or about 10% w/w based on the total weight of the composition.

In an aspect of the present application, zileuton is present in a micronized state in the topical composition of the present application. Zileuton is subjected to high-pressure homogenization to reduce its particle size. The topical composition of the present application comprises zileuton having a D90 particle size of from about 50 microns to about 1 micron.

In an aspect of the present application, the topical composition of the present application comprises zileuton having a D90 particle size of less than about 40 microns.

In an aspect of the present application, the topical composition of the present application comprises zileuton having a D90 particle size of less than about 30 microns.

In an aspect of the present application, the topical composition of the present application comprises zileuton having a D90 particle size of less than about 20 microns.

In an aspect of the present application, the topical composition of the present application comprises zileuton having a D90 particle size of less than about 10 microns.

In an aspect of the present application, the topical composition of the present application comprises zileuton having a D90 particle size of less than about 5 microns.

In an aspect of the present application, the topical composition of the present application comprises zileuton having a D90 particle size of about 5 microns.

In an aspect of the present application, the topical composition of the present application comprises zileuton having a D90 particle size of about 3 microns.

In an aspect of the present application, the topical composition of the present application comprises zileuton having a D90 particle size from about 0.1 microns to about 10 microns.

In an aspect of the present application, the topical composition of the present application comprises zileuton having D90 particle size of about 0.1 micron, or about 0.25 micron, or about 0.5 micron, or about 0.75 micron, or about 1 micron, or about 1.25 micron, or about 1.5 micron, or about 1.75 micron, or about 2 micron, or about 2.25 micron, or about 2.5 micron, or about 2.75 micron, or about 3 micron, or about 3.25 micron, or about 3.5 micron, or about 3.75 micron, or about 4 micron, or about 4.25 micron, or about 4.5 micron, or about 4.75 micron, or about 5 micron, or about 5.25 micron, or about 5.5 micron, or about 5.75 micron, or about 6 micron, or about 6.25 micron, or about 6.5 micron, or about 6.75 micron, or about 7 micron, or about 7.25 micron, or about 7.5 micron, or about 7.75 micron, or about 8 micron, or about 8.25 micron, or about 8.5 micron, or about 8.75 micron, or about 9 micron, or about 9.25 micron, or about 9.5 micron, or about 9.75 micron, or about 10 micron.

An aspect of the present application relates to a topical composition comprising zileuton; wherein zileuton is dispersed in the said composition.

In some aspects of the present application, the topical composition comprising zileuton is monophasic.

In some aspects of the present application, the topical composition comprising zileuton is a biphasic composition such as emulsion.

In some aspects of the present application, the topical composition comprising zileuton is an anhydrous biphasic composition.

In an aspect of the present application, the topical composition comprising zileuton is an emulsion base; wherein the water content is in an amount of less than about 50% w/w based on the total weight of the composition.

In some aspects of the present application, the topical composition comprising zileuton is an emulsion base; wherein the water content is in an amount of less than about 40% w/w based on the total weight of the composition.

In some aspects of the present application, the topical composition comprising zileuton is an emulsion base; wherein the water content is in an amount of less than about 30% w/w based on the total weight of the composition.

In some aspects of the present application, the topical composition comprising zileuton is an emulsion base; wherein the water content is in an amount of less than about 20% w/w based on the total weight of the composition.

In some aspects of the present application, the topical composition comprising zileuton is an emulsion base; wherein the water content is in an amount of less than about 10% w/w based on the total weight of the composition.

In some aspects of the present application, the topical composition comprising zileuton comprises in an amount of less than about 5% w/w of water based on the total weight of the composition.

In some aspects of the present application, the topical composition comprising zileuton comprises in an amount of less than about 4% w/w of water based on the total weight of the composition.

In some aspects of the present application, the topical composition comprising zileuton comprises in an amount of less than about 3% w/w of water based on the total weight of the composition.

In some aspects of the present application, the topical composition comprising zileuton comprises in an amount of less than about 2% w/w of water based on the total weight of the composition.

In some aspects of the present application, the topical composition comprising zileuton comprises in an amount of less than about 1% w/w of water based on the total weight of the composition.

In some aspects of the present application, the topical composition comprising zileuton comprises in an amount of about 0% w/w of water based on the total weight of the composition.

In some aspects of the present application, the topical composition comprising zileuton comprises 0% w/w of water based on the total weight of the composition.

In some aspects of the present application, the topical composition comprising zileuton is substantially anhydrous.

In some aspects of the present application, the topical composition comprising zileuton is anhydrous.

In some aspects of the present application, the topical composition comprising zileuton is substantially free of water-miscible excipient(s).

In some aspects of the present application, the topical composition comprising zileuton is substantially free of hyaluronic acid.

In some aspects of the present application, the topical composition comprising zileuton is free of hyaluronic acid.

In some aspects of the present application, the topical composition comprising zileuton is substantially free of a free of lower alcohol (C1-C5).

In some aspects of the present application, the topical composition comprising zileuton is free of a free of lower alcohols (C1-C5).

In some aspects of the present application, the topical compositions are propellant-free.

In some aspects of the present application, the topical compositions are non-foaming, i.e., not delivered as foam on the skin or at the application site.

In some aspects of the present application, the topical composition comprising zileuton is substantially free of linear or branched lower alcohols, such as methyl alcohol, ethyl alcohol, propyl alcohol, isopropyl alcohol, butyl alcohol, and pentyl alcohol.

In some aspects of the present application, the topical composition comprising zileuton is substantially free of methyl alcohol, ethyl alcohol, and isopropyl alcohol.

In some aspects of the present application, the topical composition comprising zileuton is substantially free of hydrophilic solvent(s).

In some aspects of the present application, the topical composition comprising zileuton is free of hydrophilic solvent(s).

The conventional cream and ointment bases containing propylene glycol are irritating to the skin, particularly over the long exposure that is frequently required for efficacy.

In an aspect of the present application, the topical composition comprising zileuton is substantially free of propylene glycol.

In an aspect of the present application, the topical composition comprising zileuton is free of propylene glycol.

In an aspect of the present application, the topical composition comprising zileuton prevents water loss from the skin of the subject when topically applied to the affected area.

In an aspect of the present application, the topical composition comprising zileuton is occlusive.

In an aspect of the present application, the composition comprising zileuton is present in the dosage form selected from, but are not limited to, gel, cream, lotion, spray, ointment, suspension, aerosol, emulsion, paste, foam, or any other mixtures thereof.

In a further aspect of the present application, the topical composition comprising zileuton is an ointment.

In some aspects of the present application, the topical composition comprises crisaborole as an active agent.

In some aspects of the present application, the topical composition comprises doxepin as an active agent.

In some aspects of the present application, the topical composition comprises tacrolimus as an active agent.

In some aspects of the present application, the topical composition comprises corticosteroid as an active agent.

The present application relates to a topical composition comprising (a) an active agent and (b) an oleaginous base; wherein the composition has an oleaginous base in an amount of at least about 60% w/w based on the total weight of the composition, and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

Some aspects of the present application relates to a topical composition comprising (a) an active agent and (b) an oleaginous base; wherein the composition has an oleaginous base in an amount of at least about 70% w/w based on the total weight of the composition, and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

The present application relates to a topical composition comprising (a) an active agent and (b) an emulsion base comprising an oil phase containing an oleaginous vehicle, and an aqueous phase containing water and/or one or more water-miscible substance(s); wherein the emulsion base has a weight ratio of aqueous phase to oil phase of about 1:2 to about 1:8. In some embodiments, the weight ratio of aqueous phase to oil phase is about 1:2, 1:3, 1:4, 1:5, 1:6, 1:7, 1:8, or 1:9

In some aspects of the present application, the water-miscible substance is in an amount of less than about 20% w/w or in an amount of less than about 20% w/w or in an amount of less than about 15% w/w or in an amount of less than about 10% w/w or in an amount of less than about 5% w/w based on the total weight of the composition.

An aspect of the present application relates to a topical composition comprising a) an active agent b) emulsion base comprising an oil phase and an aqueous phase; wherein the emulsion base is free of pH adjusting agent(s), and buffer(s); wherein the active agent is present in a non-solubilized form in the composition.

In some aspects of the present application, the oil phase comprises one or more water-immiscible substance(s) selected from fatty alcohol(s), fatty acid(s), ethers of fatty alcohol(s), esters of fatty acid(s), terpenes, mineral oil, soft paraffin, hard paraffin, petrolatum, mixture of mineral oil and lanolin alcohols, coconut oil, almond oil, lanolin, mixture of petrolatum and lanolin alcohols, vegetable oils, and mixtures thereof.

In some aspects of the present application, the aqueous phase of emulsion base comprises water and optionally one or more water-miscible excipient(s) such as polymers, preservatives, anti-oxidants, emollient(s), and the like.

In some aspects of the present application, the emulsion base of the topical composition is substantially free of propylene glycol, glycol ether(s), and lower alcohol such as ethanol.

In some aspects of the present application, the emulsion base of the topical composition is free of propylene glycol, glycol ether(s), and lower alcohol such as ethanol.

In an aspect of the present application, the topical composition has a viscosity from about 1 centipoise to about 200000 centipoises.

In an aspect of the present application, the topical composition has a viscosity ranging from about 100 cps to about 10000 cps when measured using Brookfield viscometer CAP2000⁺, spindle number 01, 20-25 rpm, and at a temperature of 25° C.±5° C.

In an aspect of the present application, the topical composition has a viscosity ranging from about 2000 cps to about 5500 cps when measured using Brookfield viscometer CAP2000+, spindle number 01, 20-25 rpm, and at a temperature of 25° C.±5° C.

In an aspect of the present application, the topical composition has a viscosity ranging from about 2400 cps to about 5000 cps when measured using Brookfield viscometer CAP2000+, spindle number 01, 20-25 rpm, and at a temperature of 25° C.±5° C.

In an aspect of the present application, the topical composition has a viscosity ranging from about 8000 cps to about 35000 cps when measured using Brookfield viscometer DV2TRV, spindle number CP-52, 2-10 rpm, and at a temperature of 25° C.±5° C.

In an aspect of the present application, the topical composition has a viscosity ranging from about 10000 cps to about 35000 cps when measured using Brookfield viscometer DV2TRV, spindle number CP-52, 2-10 rpm, and at a temperature of 25° C.±5° C.

In an aspect of the present application, the topical composition is physically and chemically stable.

In an aspect of the present application, the topical composition is stable at least for about 12 months at room temperature.

The present application relates to a topical composition comprising crisaborole.

An aspect of the present application relates to a topical composition comprising crisaborole and an oleaginous base.

An aspect of the present application relates to a topical composition comprising crisaborole and an oleaginous base; wherein the oleaginous base is substantially free of water.

An aspect of the present application relates to a topical composition comprising crisaborole and an oleaginous base; wherein the oleaginous base comprises an oleaginous vehicle in an amount of at least about 60% w/w based on the total weight of the composition.

An aspect of the present application relates to a topical composition comprising crisaborole and an oleaginous base; wherein the oleaginous base comprises a skin penetration enhancer in an amount of less than about 20% w/w based on the total weight of the composition.

An aspect of the present application relates to a topical composition comprising crisaborole and an oleaginous base; wherein the oleaginous base comprises a skin penetration enhancer and an oleaginous vehicle in an amount of at least about 60% w/w based on the total weight of the composition; wherein the skin penetration enhancer is in the form of a liquid at room temperature.

An aspect of the present application relates to a topical composition comprising crisaborole and an oleaginous base; wherein the oleaginous base comprises (i) one or more stiffening agent(s), (ii) a skin penetration enhancer and (iii) an oleaginous vehicle having melting point more than about 35° C.; wherein the weight ratio between the skin penetration enhancer and the oleaginous vehicle is in the range of from about 1:5 to about 1:9.5. In some embodiments, the weight ratio between the skin penetration enhancer and the oleaginous vehicle is about 2:7, 1:5, 2:11, 1:6, 1:13, 1:7, 2:15, 1:8, 2:17, 1:9, 2:19, 1:10, 1:11, 1:12, 1:13, 1:14, 1:15, 1:16, 1:17, and 1:18.

The present application relates to a topical composition comprising (a) crisaborole and (b) an oleaginous base; wherein the composition has an oleaginous base in an amount of at least about 60% w/w based on the total weight of the composition, and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

Some aspects of the present application relate to a topical composition comprising (a) crisaborole and (b) an oleaginous base; wherein the composition has an oleaginous base in an amount of at least about 70% w/w based on the total weight of the composition, and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

The present application relates to a topical composition comprising (a) crisaborole and (b) an emulsion base comprising an oil phase containing an oleaginous vehicle, and an aqueous phase containing water and/or one or more water-miscible substance(s); wherein the emulsion base has a weight ratio of aqueous phase to oil phase is about 1:2 to about 1:8. In some embodiments, the weight ratio of aqueous phase to oil phase is about 1:2, 1:3, 1:4, 1:5, 1:6, 1:7, 1:8, or 1:9.

In some aspects of the present application, the water-miscible substance is in an amount of less than about 20% w/w based on the total weight of the composition.

In some aspects of the present application, the topical composition comprising crisaborole is substantially free of water.

An aspect of the present application relates to a topical composition comprising a) crisaborole b) an oleaginous base comprising an oleaginous vehicle in an amount of at least about 40% w/w based on the total weight of an oleaginous base, one or more stiffening agent(s) and c) one or more pharmaceutically acceptable excipient(s).

In some aspects of the present application, the oleaginous vehicle is white petrolatum and is present in an amount of at least about 40% w/w based on the total weight of the oleaginous base.

In some aspects of the present application, the oleaginous vehicle is white petrolatum and is present in an amount of at least about 50% w/w based on the total weight of the oleaginous base.

In some aspects of the present application, the oleaginous vehicle is white petrolatum and is present in an amount of at least about 60% w/w based on the total weight of the oleaginous base.

In some aspects of the present application, the oleaginous vehicle is white petrolatum and is present in an amount of at least about 70% w/w based on the total weight of the oleaginous base.

In some aspects of the present application, the oleaginous vehicle is paraffin and is present in an amount of at least about 40% w/w based on the total weight of the oleaginous base.

In a further aspect of the present application, crisaborole is present in an amount of about 1%, or about 1.25%, or about 1.5%, or about 1.75%, or about 2%, or about 2.25%, or about 2.5%, or about 2.75%, or about 3%, or about 3.25%, or about 3.5%, or about 3.75%, or about 4%, or about 4.25%, or about 4.5%, or about 4.75%, or about 5% based on the total weight of the composition.

An aspect of the present application relates to a topical composition comprising crisaborole and an oleaginous base; wherein the crisaborole is in non-solubilized form and having a D90 particle size less than about 50 microns.

In some aspects of the present application, the topical composition comprising crisaborole is substantially anhydrous.

The present application relates to a topical composition comprising doxepin.

An aspect of the present application relates to a topical composition comprising doxepin and an oleaginous base.

An aspect of the present application relates to a topical composition comprising doxepin and an oleaginous base; wherein the oleaginous base is substantially free of water.

An aspect of the present application relates to a topical composition comprising doxepin and an oleaginous base; wherein the oleaginous base comprises an oleaginous vehicle in an amount of at least about 60% w/w based on the total weight of the composition.

An aspect of the present application relates to a topical composition comprising doxepin and an oleaginous base; wherein the oleaginous base comprises a skin penetration enhancer in an amount of less than about 20% w/w based on the total weight of the composition.

An aspect of the present application relates to a topical composition comprising doxepin and an oleaginous base; wherein the oleaginous base comprises a skin penetration enhancer and an oleaginous vehicle in an amount of at least about 60% w/w based on the total weight of the composition; wherein the skin penetration enhancer is in the form of a liquid at room temperature.

An aspect of the present application relates to a topical composition comprising doxepin and an oleaginous base; wherein the oleaginous base comprises (i) one or more stiffening agent(s), (ii) a skin penetration enhancer and (iii) an oleaginous vehicle having melting point more than about 35° C.; wherein the weight ratio between the skin penetration enhancer and the oleaginous vehicle is in the range of from about 1:5 to about 1:9.5. In some embodiments, the weight ratio between the skin penetration enhancer and the oleaginous vehicle is about 2:7, 1:5, 2:11, 1:6, 1:13, 1:7, 2:15, 1:8, 2:17, 1:9, 2:19, 1:10, 1:11, 1:12, 1:13, 1:14, 1:15, 1:16, 1:17, and 1:18.

The present application relates to a topical composition comprising (a) doxepin and (b) an oleaginous base; wherein the composition has an oleaginous base in an amount of at least about 60% w/w based on the total weight of the composition, and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

Some aspects of the present application relate to a topical composition comprising (a) doxepin and (b) an oleaginous base; wherein the composition has an oleaginous base in an amount of at least about 70% w/w based on the total weight of the composition, and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

The present application relates to a topical composition comprising (a) doxepin and (b) an emulsion base comprising an oil phase containing an oleaginous vehicle, and an aqueous phase containing water and/or one or more water-miscible substance(s); wherein the emulsion base has a weight ratio of aqueous phase to oil phase is about 1:2 to about 1:8. In some embodiments, the weight ratio of aqueous phase to oil phase is about 1:2, 1:3, 1:4, 1:5, 1:6, 1:7, 1:8, or 1:9.

In some aspects of the present application, the water-miscible substance is in an amount of less than about 20% w/w based on the total weight of the composition.

In some aspects of the present application, the topical composition comprising doxepin is substantially free of water.

An aspect of the present application relates to a topical composition comprising a) doxepin, b) an oleaginous base comprising an oleaginous vehicle in an amount of at least about 40% w/w based on the total weight of an oleaginous base, one or more stiffening agent(s) and c) one or more pharmaceutically acceptable excipient(s).

In some aspects of the present application, the oleaginous vehicle is white petrolatum and is present in an amount of at least about 40% w/w based on the total weight of the oleaginous base.

In some aspects of the present application, the oleaginous vehicle is white petrolatum and is present in an amount of at least about 50% w/w based on the total weight of the oleaginous base.

In some aspects of the present application, the oleaginous vehicle is white petrolatum and is present in an amount of at least about 60% w/w based on the total weight of the oleaginous base.

In some aspects of the present application, the oleaginous vehicle is white petrolatum and is present in an amount of at least about 70% w/w based on the total weight of the oleaginous base.

In some aspects of the present application, the oleaginous vehicle is paraffin and is present in an amount of at least about 40% w/w based on the total weight of the oleaginous base.

In further aspects of the present application, doxepin is present in an amount of about 1%, or about 1.25%, or about 1.5%, or about 1.75%, or about 2%, or about 2.25%, or about 2.5%, or about 2.75%, or about 3%, or about 3.25%, or about 3.5%, or about 3.75%, or about 4%, or about 4.25%, or about 4.5%, or about 4.75%, or about 5% or about 5.25%, or about 5.5%, or about 5.75%, or about 6%, or about 6.25%, or about 6.5%, or about 6.75%, or about 7%, or about 7.25%, or about 7.5%, or about 7.75%, or about 8%, or about 8.25%, or about 8.5%, or about 8.75%, or about 9%, or about 9.25%, or about 9.5%, or about 9.75%, or about 10% w/w based on the total weight of the composition.

An aspect of the present application relates to a topical composition comprising doxepin and an oleaginous base; wherein the doxepin is in non-solubilized form and having a D90 particle size less than about 50 microns.

In some aspects of the present application, the topical composition comprising doxepin is substantially anhydrous.

In an aspect, the present application relates to a topical composition comprising tacrolimus.

An aspect of the present application relates to a topical composition comprising tacrolimus and an oleaginous base.

An aspect of the present application relates to a topical composition comprising tacrolimus and an oleaginous base; wherein the oleaginous base is substantially free of water.

In an aspect of the present application, the topical composition comprises water in an amount of less than about 10% w/w.

An aspect of the present application relates to a topical composition comprising tacrolimus and an oleaginous base; wherein the oleaginous base comprises an oleaginous vehicle in an amount of at least about 60% w/w based on the total weight of the composition.

An aspect of the present application relates to a topical composition comprising tacrolimus and an oleaginous base; wherein the oleaginous base comprises a skin penetration enhancer in an amount of less than about 20% w/w based on the total weight of the composition.

An aspect of the present application relates to a topical composition comprising tacrolimus and an oleaginous base; wherein the oleaginous base comprises a skin penetration enhancer and an oleaginous vehicle in an amount of at least about 60% w/w based on the total weight of the composition; wherein the skin penetration enhancer is in the form of a liquid at room temperature.

An aspect of the present application relates to a topical composition comprising tacrolimus and an oleaginous base; wherein the oleaginous base comprises (i) one or more stiffening agent(s), (ii) a skin penetration enhancer and (iii) an oleaginous vehicle having melting point more than about 35° C.; wherein the weight ratio between the skin penetration enhancer and the oleaginous vehicle is in the range of from about 1:5 to about 1:9.5. In some embodiments, the weight ratio between the skin penetration enhancer and the oleaginous vehicle is about 2:7, 1:5, 2:11, 1:6, 1:13, 1:7, 2:15, 1:8, 2:17, 1:9, 2:19, 1:10, 1:11, 1:12, 1:13, 1:14, 1:15, 1:16, 1:17, and 1:18.

An aspect of the present application relates to a topical composition comprising (a) tacrolimus and (b) an oleaginous base; wherein the composition has an oleaginous base in an amount of at least about 60% w/w based on the total weight of the composition, and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

Some aspects of the present application relate to a topical composition comprising (a) tacrolimus and (b) an oleaginous base; wherein the composition has oleaginous base in an amount of at least about 60% w/w based on the total weight of the composition, and the composition is free of hydrophilic solvent(s) selected from ethanol, isopropyl alcohol, ethylene glycol, polyethylene glycol (2 to 20 monomers), propylene glycol, dipropylene glycol, butylene glycol, pentylene glycol, and hexylene glycol.

The present application relates to a topical composition comprising (a) tacrolimus and (b) an emulsion base comprising an oil phase containing an oleaginous vehicle, and an aqueous phase containing water and/or one or more water-miscible substance(s); wherein the emulsion base has a weight ratio of aqueous phase to oil phase is about 1:2 to about 1:8. In some embodiments, the weight ratio of aqueous phase to oil phase is about 1:2, 1:3, 1:4, 1:5, 1:6, 1:7, 1:8, or 1:9.

In some aspects of the present application, the water-miscible substance is in an amount of less than about 20% w/w based on the total weight of the composition.

In some aspects of the present application, the topical composition comprising tacrolimus is substantially free of water.

An aspect of the present application relates to a topical composition comprising a) tacrolimus b) an oleaginous base comprising an oleaginous vehicle in an amount of at least about 40% w/w based on the total weight of an oleaginous base, one or more stiffening agent(s), and c) one or more pharmaceutically acceptable excipient(s).

In some aspects of the present application, the oleaginous vehicle is white petrolatum and is present in an amount of at least about 40% w/w based on the total weight of the oleaginous base.

In some aspects of the present application, the oleaginous vehicle is white petrolatum and is present in an amount of at least about 50% w/w based on the total weight of the oleaginous base.

In some aspects of the present application, the oleaginous vehicle is white petrolatum and is present in an amount of at least about 60% w/w based on the total weight of the oleaginous base.

In some aspects of the present application, the oleaginous vehicle is white petrolatum and is present in an amount of at least about 70% w/w based on the total weight of the oleaginous base.

In some aspects of the present application, the oleaginous vehicle is paraffin and is present in an amount of at least about 40% w/w based on the total weight of the oleaginous base.

In one aspect of the present invention, tacrolimus is present in an amount of about 0.01%, to about 1% w/w based on the total weight of the composition.

In one aspect of the present application relates to a topical composition comprising tacrolimus and an oleaginous base; wherein the tacrolimus is in non-solubilized form and having a D90 particle size less than about 50 microns.

In an aspect of the present application, the topical composition comprising tacrolimus is substantially anhydrous.

The present application relates to a process of preparing topical oleaginous composition, the process comprising steps of a) preparing an oleaginous base by melting an oleaginous vehicle with one or more excipient(s), b) cooling the oleaginous base, c) addition of active agent to the oleaginous base with homogenization for at least 15 minutes to prepare an oleaginous composition, and d) cooling of the composition at the temperature of above about 40° C.±5° C. with stirring to prepare final composition. In some aspects of the present application, the process of preparing involves step a) preparing the oleaginous base by melting an oleaginous vehicle with one or more excipient(s) at the temperature of above about 60° C.

In an aspect of the present application, the process of preparing the topical composition involves cooling the oleaginous base at the temperature of above about 50° C.

In some aspects of the present application, the process of preparing the topical composition involves addition of the active agent to the oleaginous base with homogenization for at least 15 minutes; wherein the addition of the active agent is in the form of powder of active agent or the form of a suspension comprising the active agent, the penetration enhancer, and optionally one or more water-immiscible substance(s).

In some aspects of the present application, the process of preparing the topical composition involves the homogenization at 6000±500 rpm for 30 minutes.

In some aspects of the present application, the process of preparing the topical composition involves addition of the active agent to the oleaginous base in the form of suspension with homogenization; wherein the suspension of the active agent is prepared by mixing active agent with penetration enhancer and petrolatum in a weight ratio selected from 1:1, 2:1, 3:1, and 4:1.

In some aspects of the present application, the process of preparing the topical composition involves cooling of the composition at the temperature of above about 35° C.±5° C.

In some aspects of the present application, the process of preparing the topical composition involves preparing the said composition on a large scale that is more than about 5 kilograms.

In some aspects of the present application, the process of preparing the topical composition is in accordance with the “Guidelines for current Good Manufacturing Practice standards for drug manufacture.”

In some aspects of the present application, the process of preparing the topical composition involves a topical composition comprising the active agent is in non-solubilized form.

In an aspect of the present application, the topical composition is prepared by a process described in Example 1.

In some aspects of the present application, the process of preparing the oleaginous composition; wherein the active agent is zileuton and is present in the non-solubilized form.

An aspect of the present application relates to a method of treating and/or preventing skin disorder(s), by topically administering a composition comprising a therapeutically effective amount of an active agent to a subject in need thereof.

An aspect of the present application relates to a method of treating and/or preventing inflammatory skin disorder(s), by topically administering a composition comprising a therapeutically effective amount of a non-steroidal active agent to a subject in need thereof.

In some aspects of the present application, the topical administration is at least one day to about twelve weeks, once or twice daily at the affected area.

In some aspects of the present application, the topical administration is at least one day to about eight weeks, once or twice daily at the affected area.

In some aspects of the present application, the topical administration is at least one day to about four weeks, once or twice daily at the affected area.

An aspect of the present application relates to a method of treating and/or preventing atopic dermatitis, by topically administering a composition comprising a therapeutically effective amount of a non-steroidal active agent to a subject in need thereof; wherein the non-steroidal active agent is selected from the group consisting of zileuton, crisaborole, tacrolimus, doxepin, and combinations thereof.

As aspect of the present application relates to a method of treating and/or preventing a skin disorder.

In an aspect of the present application, the skin disorder is inflammatory skin disorder.

In some aspects of the present application, the skin disorder is selected from acne, psoriasis, rosacea, sebaceous disorder(s), plaque psoriasis, atopic dermatitis, contact dermatitis, seborrheic dermatitis, pruritus of any skin disorder, allergic dermatitis, actinic keratosis, eczema, basal cell carcinoma, keloids, melanoma, scleroderma, sebaceous carcinoma, seborrheic keratosis, seborrheic keratosis, vitiligo, warts, and the like.

In some aspects of the present application, the skin disorder is atopic dermatitis.

In some aspects of the present application, the skin disorder is mild to moderate atopic dermatitis.

In some aspects of the present application, the skin disorder is pruritus of severe atopic dermatitis.

In some aspects of the present application, the skin disorder is pruritus of a skin disorder.

In some aspects of the present application, the skin disorder is pruritus of atopic dermatitis.

An aspect of the present application relates to a method of treating and/or preventing atopic dermatitis, by topically administering a composition comprising a therapeutically effective amount of zileuton, its pharmaceutically acceptable salt, prodrug, ester, solvate, or polymorph thereof.

Some aspects of the present application relate to a method of treating and/or preventing pruritus, by topically administering a composition comprising a therapeutically effective amount of zileuton, its pharmaceutically acceptable salt, prodrug, ester, solvate, or polymorph thereof.

An aspect of the present application relates to a topical composition comprising a non-steroidal active agent selected from zileuton, crisaborole, tacrolimus, and doxepin for the treatment of a skin disorder(s) in a subject by topically administering a composition comprising a therapeutically effective amount of the active agent; wherein the composition is administered for at least one day; wherein the composition is an oleaginous composition comprising in an amount of at least about 60% w/w of an oleaginous vehicle.

Some aspects of the present application relate to a method of treating and/or preventing a skin disorder; wherein the method comprises topically administering a composition comprising zileuton to a subject, and the composition is an oleaginous composition comprising an oleaginous vehicle in an amount of at least about 60% w/w based on the total weight of the composition.

Some aspects of the present application relate to a topical composition comprising a zileuton for the treatment of a skin disorder(s) in a subject by topically administering a composition comprising a therapeutically effective amount of zileuton; wherein the composition is administered for at least one day; wherein the composition is an oleaginous composition comprising in an amount of at least about 60% w/w of an oleaginous vehicle.

In some aspects of the present application, the skin disorder is inflammatory skin disorder selected from acne, psoriasis, rosacea, dermatitis, atopic dermatitis, contact dermatitis, seborrheic dermatitis, pruritus of any skin disorder and combinations thereof.

An aspect of the present application relates to a method of treating and/or preventing a skin disorder in a subject; wherein the method comprises administering topically an oleaginous composition comprising zileuton for at least one day to four weeks once or twice daily.

An aspect of the present application relates to a method of treating and/or preventing a skin disorder in a subject; wherein the method comprises administering topically an oleaginous composition comprising zileuton for at least one day to eight weeks once or twice daily.

An aspect of the present application relates to a method of treating and/or preventing a skin disorder in a subject; wherein the method comprises administering topically an oleaginous composition comprising zileuton for at least one day to twelve weeks once or twice daily.

An aspect of the present application relates to a method of treating and/or preventing a skin disorder in a subject; wherein the method comprises administering topically an oleaginous composition comprising crisaborole for at least one day to four weeks once or twice daily.

An aspect of the present application relates to a method of treating and/or preventing a skin disorder in a subject; wherein the method comprises administering topically an oleaginous composition comprising doxepin for at least one day to four weeks once or twice daily.

An aspect of the present application relates to a method of treating and/or preventing a skin disorder in a subject; wherein the method comprises administering topically an oleaginous composition comprising tacrolimus for at least one day to four weeks once or twice daily.

One aspect of the present application relates to a method of treating and/or preventing a skin disorder in a subject; wherein the method comprises topically administering an oleaginous composition comprising crisaborole for at least one day to four weeks once or twice daily; wherein the composition comprises an oleaginous vehicle in an amount of at least about 60% w/w based on the total weight of the composition.

One aspect of the present application relates to a method of treating and/or preventing a skin disorder in a subject; wherein the method comprises topically administering an oleaginous composition comprising doxepin for at least one day to four weeks once or twice daily; wherein the composition comprises an oleaginous vehicle in an amount of at least about 60% w/w based on the total weight of the composition.

One aspect of the present application relates to a method of treating and/or preventing a skin disorder in a subject; wherein the method comprises topically administering an oleaginous composition comprising tacrolimus for at least one day to four weeks once or twice daily; wherein the composition comprises an oleaginous vehicle in an amount of at least about 60% w/w based on the total weight of the composition.

One aspect of the present application relates to a method of improving the success rate in treating and/or preventing skin disorder(s) using a topical composition, as disclosed herein.

One aspect of the present application relates to a method of improving the success rate in treating and/or preventing skin disorder(s), with at least a two-grade reduction from the baseline to 4 weeks as compared to vehicle, by topically administering a composition comprising a therapeutically effective amount of zileuton to a subject in need thereof.

An aspect of the present application relates to a method of improving the success rate in treating and/or preventing skin disorder(s), with at least a two-grade reduction from the baseline to 8 weeks as compared to the vehicle, by topically administering a composition comprising a therapeutically effective amount of zileuton to a subject in need thereof.

An aspect of the present application relates to a method of improving the success rate in treating and/or preventing skin disorder(s), with at least a two-grade reduction from the baseline to 12 weeks as compared to the vehicle, by topically administering a composition comprising a therapeutically effective amount of zileuton to a subject in need thereof.

An aspect of the present application relates to a method of improving the success rate in treating and/or preventing skin disorder(s), with a 5-point reduction in DLQI score from baseline to 1 week, by topically administering a composition comprising a therapeutically effective amount of zileuton to a subject in need thereof.

An aspect of the present application relates to a method of improving the success rate in treating and/or preventing skin disorder(s,) with a 5-point reduction in DLQI score from baseline to 2 weeks, by topically administering a composition comprising a therapeutically effective amount of zileuton to a subject in need thereof.

An aspect of the present application relates to a method of improving the success rate in treating and/or preventing skin disorder(s), with a 5-point reduction in DLQI score from baseline to 4 weeks, by topically administering a composition comprising a therapeutically effective amount of zileuton to a subject in need thereof.

An aspect of the present application relates to a method of treating and/or preventing pruritus by topically administering a composition comprising a therapeutically effective amount of zileuton to a subject in need thereof, wherein the composition when topically administered provides at least 60% inhibition of itching/scratching behavior in mice as compared to placebo.

An aspect of the present application relates to a method of treating and/or preventing pruritus by topically administering a composition comprising a therapeutically effective amount of zileuton to a subject in need thereof, wherein the composition when topically administered provides at least 40% inhibition of itching/scratching frequency in mice on day 14 as compared to vehicle.

An aspect of the present application relates to a method of administering a topical composition comprising an active agent, wherein the method comprises administering a thin layer of composition once or twice for at least one day at affected area of the skin in a subject in need thereof, and the composition comprises an oleaginous vehicle in an amount of at least about 60% w/w based on the total weight of the composition.

An aspect of the present application relates to a method of administering a topical composition comprising zileuton, wherein the method comprises administering a thin layer of composition once or twice for at least one day at affected area of the skin in a subject in need thereof, and the composition comprises zileuton from about 0.1% w/w to about 10% w/w; and an oleaginous vehicle in an amount of at least about 60% w/w based on the total weight of the composition.

An aspect of the present application relates to a method of administering a topical composition comprising crisaborole, wherein the method comprises administering a thin layer of composition once or twice for at least one day at affected area of the skin in a subject in need thereof, and the composition comprises crisaborole from about 0.1% w/w to about 10% w/w, and an oleaginous vehicle in an amount of at least about 60% w/w based on the total weight of the composition.

An aspect of the present application relates to a method of administering a topical composition comprising doxepin, wherein the method comprises administering a thin layer of composition once or twice for at least one day at affected area of the skin in a subject in need thereof, and the composition comprises doxepin from about 0.1% w/w to about 10% w/w, and an oleaginous vehicle in an amount of at least about 60% w/w based on the total weight of the composition.

An aspect of the present application relates to a method of administering a topical composition comprising tacrolimus, wherein the method comprises administering a thin layer of composition once or twice for at least one day at affected area of the skin in a subject in need thereof, and the composition comprises tacrolimus from about 0.1% w/w to about 10% w/w, and an oleaginous vehicle in an amount of at least about 60% w/w based on the total weight of the composition.

An aspect of the present application relates to a method of administering a topical composition, wherein the method comprises forming a thin oily film at the application site, and it reduces the water loss, moisturizes the area, and provides emollient effect at the application site.

In an aspect of the present application, the topical composition comprises a skin penetration enhancer and an oleaginous vehicle in an amount of at least about 60% w/w based on the total weight of the composition.

In an aspect of the present application, the topical composition comprising an active agent selected from zileuton, crisaborole, doxepin, tacrolimus, betamethasone, clobetasol, halobetasol, mometasone, dexamethasone and any combination thereof, a skin penetration enhancer and an oleaginous vehicle in an amount of at least about 60% w/w based on the total weight of the composition for treating and/or preventing a skin disorder; wherein the composition is administered at least one day until four weeks, and the skin disorder is dermatitis.

In some aspects of the present application, the skin disorder is atopic dermatitis.

In some aspects of the present application, the skin disorder is mild to moderate atopic dermatitis.

In some aspects of the present application, the skin disorder is pruritus of severe atopic dermatitis.

In some aspects of the present application, the skin disorder is pruritus of a skin disorder.

In some aspects of the present application, the skin disorder is pruritus of atopic dermatitis.

The following examples are provided to illustrate certain specific aspects and embodiments of the application and are not to be construed as limiting the scope of the invention in any manner.

### EXAMPLES

### Example 1

### Topical Oleaginous Compositions

TABLE 1

| | Example 1 (% w/w) | | | | | | | | |
|---|---|---|---|---|---|---|---|---|---|
| Ingredients | 1A | 1B | 1C | 1D | 1E | 1F | 1G | 1H | 1J |
| Zileuton | 5 | 5 | 5 | 5 | 5 | 0 | 0 | 1.25 | 2.5 |
| Crisaborole | 0 | 0 | 0 | 0 | 0 | 5 | 0 | 0 | 0 |
| Doxepin | 0 | 0 | 0 | 0 | 0 | 0 | 5 | 0 | 0 |
| Isopropyl myristate | 0 | 0 | 10 | 15 | 15 | 10 | 10 | 10 | 10 |
| Oleyl alcohol | 10 | 0 | 0 | 0 | 0 | 0 | 0 | 0 | 0 |
| Oleic acid | 0 | 25 | 0 | 0 | 0 | 0 | 0 | 0 | 0 |
| Microcrystalline wax | 0 | 0 | 10 | 5 | 5 | 10 | 10 | 10 | 10 |
| White wax | 0 | 0 | 5 | 5 | 0 | 5 | 5 | 5 | 5 |
| Methylparaben | 0 | 0 | 0.3 | 0.3 | 0 | 0 | 0 | 0 | 0 |
| Butylated | 0.1 | 0.1 | — | 0.2 | 0.2 | 0 | 0 | 0 | 0 |
| hydroxytoluene | | | | | | | | | |
| White petrolatum | 84.9 | 69.9 | 69.7 | 69.5 | 74.8 | 70 | 70 | 73.75 | 72.5 |

Process of preparation:
1. Microcrystalline wax and/or white wax and/or white petrolatum were weighed and were heated between 60° C.±5° C. and 80° C.±5° C. in a manufacturing vessel and stirred till they completely melt down,
2. Isopropyl myristate, and/or oleyl alcohol and/or oleic acid, and optionally butylated hydroxy toluene, and methylparaben were weighed and heated in at 50° C. in a separate stainless steel vessel till their dissolution,
3. Dissolution obtained from Step ii was added in Step i, melted petrolatum bulk and the mixture was cooled between 50° C.±2° C. and 65° C.±2° C. under continuous stirring at 150-250 rpm,
4. The active agent (zileuton, or crisaborole, or doxepin) was then dispersed in the above mixture under homogenization at 5000±1000 rpm for 30 mins. The temperature of the dispersed mixture was maintained below 65° C., and
5. The formulation was cooled under the continuous stirring to 40° C.±5° C.

The prepared composition of Example 1C was exposed to different stability testing conditions. The results of the stability studies are tabulated in Table 2.

TABLE 2

| | Assay of | The related substance of zileuton | | | | | | | |
|---|---|---|---|---|---|---|---|---|---|
| Testing condition | Zileuton | Comp. D | Comp. E | Comp. A | Comp. G | Comp. C | Comp. H | Unknown | Total |
| Initial | 99.2 | ND | 0.231 | ND | ND | ND | BQL | NA | 0.251 |
| 1M25° C./60% RH | 102.2 | ND | 0.079 | ND | ND | ND | ND | NA | 0.079 |
| 1M40° C./75% RH | 100.7 | ND | 0.108 | ND | ND | ND | ND | NA | 0.108 |
| 2M 25° C./60% RH | 98 | ND | 0.101 | ND | ND | ND | ND | NA | 0.101 |
| 2M 40° C./75% RH | 98.7 | ND | 0.107 | ND | ND | ND | ND | NA | 0.107 |
| 3M 25° C./60% RH | 99.4 | ND | 0.128 | ND | ND | ND | ND | NA | 0.128 |
| 3M 40° C./75% RH | 100.7 | ND | 0.182 | ND | ND | BQL | ND | NA | 0.225 |
| 6M 25° C./60% RH | 98.4 | ND | 0.272 | ND | ND | BQL | ND | BQL | 0.293 |
| 6M 30° C./65% RH | 101.1 | ND | 0.229 | ND | ND | BQL | ND | BQL | 0.257 |
| 6M 40° C./75% RH | 101 | ND | 0.287 | ND | ND | BQL | ND | BQL | 0.393 |
| 9M 25° C./60% RH | 97.6 | ND | 0.16 | ND | ND | 0.07 | ND | ND | 0.24 |
| 9M 30° C./65% RH | 89.6 | ND | 0.21 | ND | ND | 0.09 | ND | ND | 0.3 |
| 12M25° C./60% RH | 100.2 | ND | 0.12 | ND | ND | ND | ND | ND | 0.12 |
| 12M30° C./65% RH | 100.1 | ND | 0.12 | ND | ND | BQL | ND | BDL | 0.12 |

*ND = not detected UA = under analysis BQL = below the quantifiable limit

### Viscosity and Content Uniformity of Example 1C

The oleaginous composition of Example 1C was evaluated for changes in viscosity and content uniformity over 12 months at various stability conditions of ICH guidelines. Viscosity was evaluated by using a Brookfield viscometer model: CAP2000⁺, spindle number 01, 20-25 rpm, at a temperature of about 25° C., run time-30 sec, and hold time 20 sec.

| | | Content uniformity | | |
|---|---|---|---|---|
| Testing condition | Viscosity (cps) | Top | Middle | Bottom |
| Initial | 4144 | NA | NA | NA |
| 1 M 25° C./60% RH | 3638 | NA | NA | NA |
| 1 M 40° C./75% RH | 3975 | NA | NA | NA |
| 2 M 25° C./60% RH | 4050 | NA | NA | NA |
| 2 M 40° C./75% RH | 4041 | NA | NA | NA |
| 3 M 25° C./60% RH | 4284 | 100.7 | 99.4 | 99.8 |
| 3 M 40° C./75% RH | 4134 | NA | NA | NA |
| 6 M 25° C./60% RH | 4144 | 99.7 | 100.8 | 99.5 |
| 6 M 30° C./65% RH | 4837 | NA | NA | NA |
| 6 M 40° C./75% RH | 4835 | NA | NA | NA |
| 9 M 25° C./60% RH | 4828 | NA | NA | NA |
| 9 M 30° C./65% RH | NA | NA | NA | NA |
| 12 M 25° C./60% RH | NA | NA | NA | NA |
| 12 M 30° C./65% RH | NA | 96 | 101.1 | 100.1 |

In one aspect of the present application, Example 1C composition did not show a change in viscosity and/or content uniformity during 12 months.

### Example 2

### Topical Oleaginous Compositions Comprising Zileuton

TABLE 3

Example 2 (% w/w)

| Ingredients | 2A | 2B | 2C | 2D |
|---|---|---|---|---|
| Zileuton | 5 | 5 | 5 | 5 |
| Isopropyl myristate | 15 | 15 | 15 | 15 |
| Butylated | 0.2 | 0.2 | 0.2 | 0.2 |
| hydroxytoluene | | | | |
| White Petrolatum | 69.5 | 64.5 | 69.5 | 64.5 |
| Microcrystalline wax | 5 | 10 | 5 | 10 |
| White wax | 5 | 5 | 5 | 5 |
| Methylparaben | 0.3 | 0.3 | 0.3 | 0.3 |

The composition of Example 2 is prepared according to the process similar to that of Example 1. These compositions were prepared with a higher percentage of isopropyl myristate and observed for physical stability. The potential syneresis (phase separation) was observed as the percentage of skin penetration enhancer increased.

### Example 3

### Topical Oleaginous Compositions Comprising Zileuton

TABLE 4

| | Example 3 (% w/w) | | | | | | | | | | |
|---|---|---|---|---|---|---|---|---|---|---|---|
| Ingredients | 3A | 3B | 3C | 3D | 3E | 3F | 3G | 3H | 3I | 3J | 3K |
| Zileuton | 5 | 5 | 5 | 5 | 5 | 5 | 5 | 5 | 5 | 5 | 5 |
| Medium-chain | 0 | 10 | 0 | 0 | 0 | 0 | 0 | 0 | 0 | 0 | 0 |
| triglycerides | | | | | | | | | | | |
| Hard paraffin | 0 | 0 | 10 | 0 | 0 | 0 | 0 | 0 | 0 | 0 | 0 |
| Myristyl alcohol | 0 | 0 | 0 | 10 | 0 | 0 | 0 | 0 | 0 | 0 | 0 |
| Dibutyl sebacate | 0 | 0 | 0 | 0 | 10 | 0 | 0 | 0 | 0 | 0 | 0 |
| Octyl Dodeconol | 0 | 0 | 0 | 0 | 0 | 10 | 0 | 0 | 0 | 0 | 0 |
| Almond oil | 0 | 0 | 0 | 0 | 0 | 0 | 10 | 0 | 0 | 0 | 0 |
| Stearic acid | 0 | 0 | 0 | 0 | 0 | 0 | 0 | 10 | 0 | 0 | 0 |
| Cyclomethicone | 0 | 0 | 0 | 0 | 0 | 0 | 0 | 0 | 10 | 0 | 0 |
| Ceresin wax | 0 | 0 | 0 | 0 | 0 | 0 | 0 | 0 | 0 | 10 | 0 |
| Cetyl ester wax | 0 | 0 | 0 | 0 | 0 | 0 | 0 | 0 | 0 | 0 | 10 |
| Butylated | 0.2 | 0.2 | 0 | 0 | 0 | 0 | 0 | 0 | 0 | 0 | 0 |
| hydroxytoluene | | | | | | | | | | | |
| White Petrolatum | 89.5 | 74.5 | 70 | 70 | 70 | 70 | 70 | 70 | 70 | 70 | 70 |
| Microcrystalline wax | 5 | 10 | 10 | 10 | 10 | 10 | 10 | 10 | 10 | 10 | 10 |
| White wax | 0 | 0 | 5 | 5 | 5 | 5 | 5 | 5 | 5 | 5 | 5 |
| Methylparaben | 0.3 | 0.3 | 0 | 0 | 0 | 0 | 0 | 0 | 0 | 0 | 0 |

The compositions of Examples 3A-3K were manufactured according to the process as defined in Example 1. Topical compositions that were prepared with various skin penetration enhancers or liquid water-immiscible and all manufactured compositions were physically stable.

### Example 4

### Topical Composition Comprising Zileuton (Biphasic System)

TABLE 5

| | Example 4 |
|---|---|
| Ingredients | (% w/w) |
| Zileuton | 5 |
| Polysorbate 20 | 1 |
| Carbomer Homopolymer Type C | 0.5 |
| Purified Water | 40 |
| Isopropyl myristate | 15 |
| Mineral Oil | 32.75 |
| Sorbitan monooleate | 0.5 |
| Butylated hydroxytoluene | 0.1 |
| Sodium Benzoate NF | 0.15 |
| Purified Water | 5 |

Process of manufacturing the composition of Example 4: The oil phase was prepared by the following method: In the main manufacturing vessel, 0.2 g of BHT was dissolved in 30 g isopropyl myristate. To this, 1 g of sorbitan monooleate was added and mixed manually to form a clear solution. To the above content, 65.5 g of mineral oil was added and mixed manually to form a clear solution. To the above content, zileuton was added under homogenization at 4000 rpm and continued homogenization for 20 min. The aqueous phase was prepared by adding 70 g of purified water in a stainless steel vessel, and Polysorbate 20, carbomer homopolymer type C was added and continued stirring for 20 min at 250-800 rpm. The oil phase and aqueous phase were emulsified under homogenization at 4000 to 5000 rpm for 20 minutes, and sodium benzoate solution in water was added to the above emulsion during homogenization.

In vitro release test was performed for the composition of Example 4 using a Franz diffusion cell Hanson FDC (automatic) and a cellulose nitrate 0.2μ (CN), under approximately 600 rpm, rinse volume-1.2 ml, and sample collection volume-1.2 ml. The receptor solution used was water: ethanol (40:60), v/v). 300 Mg of the oleaginous composition prepared according to Example 4 and was placed uniformly on the membrane at a temperature of 32° C.±0.5° C. (infinity dose). The amount of active agent released was determined using an HPLC method [Alltima C18, 75×4.6 mm, 3 μm), mobile phase of 0.05% TFA in 1000 ml Milli-Q water, 100% methanol, flow rate of 0.6 ml/min], column temperature was 40° C. and was analyzed using a UV detector at 230 nm. The active agent release rate at 0 minutes, 30 minutes, 60 minutes, 90 minutes, 120 minutes, 150 minutes, and 180 minutes were presented in Table 6:

TABLE 6

| Time (in | Active agent release from Example 4 composition |
|---|---|
| minutes) | (μg/cm²) |
| 30 | 622 |
| 60 | 860 |
| 90 | 1161 |
| 120 | 1427 |
| 150 | 1693 |
| 180 | 1939 |

### Example 5

### Topical Oleaginous Compositions Comprising Zileuton

TABLE 7

| | Example 5 (% w/w) | | |
|---|---|---|---|
| Ingredients | 5A | 5B | 5C |
| Zileuton | 5 | 5 | 5 |
| Isopropyl myristate | 10 | 10 | 10 |
| White Petrolatum | 79 | 65 | 70 |
| Microcrystalline wax | 3 | 10 | 10 |
| White wax | 3 | 10 | 5 |

The topical compositions described in Table 7 were prepared using the process mentioned in Example 1. These three compositions were prepared to evaluate the impact of petrolatum (oleaginous vehicle) on the in vitro release of the active agent (zileuton).

In vitro release test results of oleaginous compositions of Example 5. The result of the in vitro test is presented in Table 7.

An in vitro release test was performed for the composition of Example 5 using a Franz diffusion cell Hanson FDC (automatic) and a cellulose nitrate 0.2μ (CN), under approximately 600 rpm, rinse volume-1.2 ml, and sample collection volume-1.2 ml. The receptor solution used was water:ethanol (40:60 v/v). 300 Mg of the oleaginous composition prepared according to Example 4 and was placed uniformly on the cellulose nitrate as a membrane at a temperature of 32° C.±0.5° C. (infinity dose). The amount of active agent released was determined using an HPLC method [(Alltima C18, 75×4.6 mm, 3 μm), mobile phase of 0.05% TFA in 1000 ml Milli-Q water, 100% methanol, flow rate of 0.6 ml/min], column temperature was 40° C. and was analyzed using a UV detector at 230 nm. The active agent release rate at 0 minutes, 30 minutes, 60 minutes, 90 minutes, 120 minutes, 150 minutes, and 180 minutes were presented in Table 7:

TABLE 7

| | Active agent release | | |
|---|---|---|---|
| Time (in | (μg/cm²) | | |
| minutes) | 5A | 5B | 5C |
| 30 | 7.29 | 17.81 | 13.78 |
| 60 | 9.75 | 25.52 | 19.70 |
| 90 | 12.40 | 32.43 | 23.19 |
| 120 | 15.81 | 44.97 | 28.49 |
| 150 | 20.60 | 52.42 | 36.56 |
| 180 | 25.77 | 57.99 | 43.96 |

In one aspect, the composition of the present application provides the release of an active agent in a controller manner.

In one aspect, the composition of the present application provides the in-vitro release of the active agent from the oleaginous base was much lower than the active agent release from the cream base.

### Example 6

### Microscopy of the Composition of Example 5C

Particle size analysis of the composition of Example 5C was performed by following analysis parameters: magnification-40 μm, size range-0.7 μm to 50 μm, calibrated X (pixel/μ)-4.5102 and calibrated Y (pixel/μ)-4.5102, and analysis method-particle identification. Results of the analysis are presented below:

TABLE 8

| | Particle (μm) | Agglomerate (μm) |
|---|---|---|
| Minimum Particle size (μm) | 0.7011 | 0.9142 |
| Maximum Particle size (μm) | 7.9603 | 13.34 |
| Average particle size (μm) | 1.7061 | 1.4496 |
| Standard deviation | 0.5794 | 1.4496 |
| Average aspect ratio | 1.6485 | 2.8742 |
| Count | 29735 | 50554 |

TABLE 9

| | Particle size (μm) | With agglomerate (μm) |
|---|---|---|
| D10 | 1.131 | 1.1305 |
| D50 | 1.614 | 1.6886 |
| D90 | 2.449 | 2.9581 |

In one embodiment, the composition of the present application provides D90 Particle size distribution of about 2.5 μm and with agglomeration about 3 μm.

### Example 7

### Topical Zileuton Composition (Biphasic)

TABLE 10

| Ingredients | % w/w |
|---|---|
| Zileuton | 5 |
| Isopropyl myristate | 15 |
| White petrolatum | 13.5 |
| Polysorbate 80 | 5 |
| Butylated hydroxytoluene | 0.1 |
| Propylparaben | 0.04 |
| Methylparaben | 0.3 |
| Polyoxyl-40-stearate | 8.8 |
| Sepineo P 600 | 2 |
| Water | 50.26 |

Process of manufacturing the composition of Example 7: Oil phase:white petrolatum was weighed and was heated at 75° C. in a manufacturing vessel till they completely meltdown, in a separate vessel isopropyl myristate, propylparaben, and butylated hydroxytoluene were weighed and heated in at 50° C. in a separate stainless steel vessel till their dissolution, and melted white petrolatum phase was mixed in isopropyl myristate phase, water as aqueous phase, polysorbate 80, polyoxyl-40-stearate and methylparaben were weighed and heated in at 75° C. in a separate stainless steel vessel and mixed till a homogenous mass is produced. Emulsification: Oil phase was added into the water phase, under homogenization, at 3000 rpm, temperature to be maintained at 70° C. for half an hour, and zileuton was then dispersed in the above mixture under homogenization at 5000±1000 rpm for 30 mins. The temperature of the dispersed mixture was maintained below 65° C. Sepineo P 600 was added into step 6, under stirring at 1000 rpm, and the formulation was cooled under continuous stirring to 35° C.±5° C.

### Example 8

### Topical Zileuton Composition (Monophasic)

TABLE 10

Example 8

| Ingredients | 8A | 8B |
|---|---|---|
| Zileuton | 5 | 5 |
| Isopropyl myristate | 5 | 5 |
| Diethylene monoethyl ether | 5 | 5 |
| Medium-chain triglycerides | 5 | 5 |
| Microcrystalline wax | 15 | 10 |
| Sorbitan Monolaurate (Span 60) | — | 5 |
| Methylparaben | 0.3 | 0.3 |
| Butylated hydroxytoluene | 0.2 | 0.2 |
| White petrolatum | 64.5 | 64.5 |

Process of manufacturing the composition of Example 8: Medium-chain triglycerides, microcrystalline wax, sorbitan monolaurate, and white petrolatum were weighed and then transferred to a stainless steel manufacturing vessel. Then heated at 75° C.±5° C. in a manufacturing vessel and stirred till they completely meltdown. In a separate vessel, isopropyl myristate, diethylene glycol monoethyl ether, butylated hydroxytoluene, and methylparaben were weighed and heated at 50° C. in a separate stainless steel vessel till a clear solution is formed. Both materials were mixed, and the mixture was cooled to 60° C.±2° C. under continuous stirring at 150-250 rpm. Zileuton was then dispersed in the above mixture under homogenization at 5000±1000 rpm for 30 mins. The temperature of the dispersed mixture was maintained below 65° C. The formulation was cooled under continuous stirring to 35° C.±5° C.

### Example 9

### Evaluation of the Effect of Zileuton in Compound 48/80 Induced Pruritus in Mice

This study was conducted to determine the anti-scratching/anti-itching effect of a topical formulation of zileuton, on Compound 48/80 induced itching/scratching behavior in Balb C mice.

Materials and Methods: Compound 48/80 is known as a mast cell degranulator for induction of allergic itching/scratching behavior in mice that directly activates signaling events characteristic of an acute allergenic response. Local application of compound 48/80 elicits short-term and predictable levels of scratching behavior in the affected area.

Pruritogen (compound 48/50) was weighed and prepared each time before the scheduled dose administration with cold saline at the concentration of 50 μg/50 μl.

The topical composition of zileuton was prepared by mixing following ingredients in the given weight ratio; dimethyl Isosorbide (5%), polyethylene glycol 400 (45%), polyethylene glycol 300 (33%), and polyethylene glycol 1450 (12%).

30 Male Balb C mice (10 mice in each group-zileuton topical and placebo topical) were acclimatized for seven days in the test room and then randomized. Approximately two days before initiation of the experiment, the rostral part of the skin on the back of each mouse was clipped. On the test day, the animals were placed into observation cages individually and acclimatized for 2 hours before the start of the experiment. Animals were dosed with zileuton topical compositions 1 hour before injection of compound 48/50 at the volume of 50 μl containing 50 μg of compound 48/50 intradermally, and mice were placed immediately back to observation cages, and bouts of scratching were measured for 30 minutes following injection.

Groups I and II—animals were administered with 5% zileuton cream and placebo cream respectively for consecutive six days (including day one treatment) without any scoring.

Results: Itching behavior in day 1: zileuton topical composition showed a significant decrease in itching/scratching behavior compared with placebo. The percentage of inhibition of itching/scratching behavior recorded for a Group I was 69.64% compared to Group II (placebo).

TABLE 11
Itching behavior on day 1

| | | Group I | | | Group II | | |
|---|---|---|---|---|---|---|---|
| Group | | (5% topical zileuton) | | | (Placebo topical) | | |
| Animal | | Body | Dose | Itching | Body | Dose | Itching |
| No. | Sex | weight | (mg) | score | weight | (mg) | score |
| 1 | M | 39.4 | 100 | 8 | 32.47 | 100 | 57 |
| 2 | M | 32.06 | 100 | 18 | 29.78 | 100 | 57 |
| 3 | M | 29.7 | 100 | 23 | 29.01 | 100 | 83 |
| 4 | M | 28.84 | 100 | 54 | 28.19 | 100 | 81 |
| 5 | M | 28.07 | 100 | 13 | 26.69 | 100 | 159 |
| 6 | M | 26.64 | 100 | 18 | 25.93 | 100 | 128 |
| 7 | M | 25.88 | 100 | 50 | 24.5 | 100 | 57 |
| 8 | M | 24.33 | 100 | 37 | 23.52 | 100 | 146 |
| 9 | M | 23.28 | 100 | 34 | 21.71 | 100 | 75 |
| 10 | M | 21.48 | 100 | 58 | 21.05 | 100 | 188 |
| | | | Average | 31.30 | | Average | 103.10 |
| | | | STD | 18.01 | | STD | 48.13 |

Itching behavior on day 7: The effect of topical zileuton on itching/scratching behavior induced by compound 48/80 at 50 μg/50 μl intradermal showed a significant decrease in itching/scratching behavior compared to placebo. The percentage inhibition of itching/scratching behavior for a Group I was 61.79% compared to Group II (placebo).

TABLE 12
Itching behavior on day 7

| | | Group I | | | Group II | | |
|---|---|---|---|---|---|---|---|
| Group | | (5% topical zileuton) | | | (Placebo topical) | | |
| Animal | | Body | Dose | Itching | Body | Dose | Itching |
| No. | Sex | weight | (mg) | score | weight | (mg) | score |
| 1 | M | 30.85 | 100 | 11 | 31.63 | 100 | 143 |
| 2 | M | 28.06 | 100 | 54 | 31.52 | 100 | 120 |
| 3 | M | 26.38 | 100 | 36 | 28.64 | 100 | 218 |
| 4 | M | 26.66 | 100 | 127 | 27.5 | 100 | 115 |
| 5 | M | 26.91 | 100 | 37 | 25.16 | 100 | 89 |
| 6 | M | 26.66 | 100 | 52 | 24.68 | 100 | 110 |
| 7 | M | 23.67 | 100 | 63 | 23.66 | 100 | 92 |
| 8 | M | 23.4 | 100 | 47 | 23.01 | 100 | 105 |
| 9 | M | 22.24 | 100 | 41 | 20.51 | 100 | 129 |
| 10 | M | 20.21 | 100 | 15 | 20.08 | 100 | 143 |
| | | | Average | 48.30 | | Average | 126.40 |
| | | | STD | 32.13 | | STD | 37.16 |

### Example 10

### Evaluation of the Effect of Zileuton in Pruritus Animal Model Comparing With PROTOPIC® Ointment

Method and material: Conventional NC/Tnd mice (10-15 weeks old) with moderate atopic dermatitis were used for the study. A total of 21 mice were grouped into three different groups (3×7 mice). Group I was 5% zileuton topical cream, Group II was a vehicle, and Group III was 0.1% PROTOPIC® ointment.

Mice in each group were topically applied twice daily to the head, neck, and dorsal skin on day 1 and 2, and once daily from day 3 to day 14. The PROTOPIC® ointment was applied once daily for 14 days.

Measurements and observations: The clinical score was measured every second day for individual mice starting before treatment and continuing during 2 weeks treatment period and clinical score was evaluated as each skin conditions such as itch, erythema, edema, excoriation/erosion, dryness/scaling, were graded as follows:

Itch: grade 0 (none); grade 1 (mild); grade 2 (moderate); grade 3 (severe); Erythema: grade 0 (none); grade 1 (mild); grade 2 (moderate); grade 3 (severe); Edema: grade 0 (none); grade 1 (mild); grade 2 (moderate); grade 3 (severe); Excoriation/erosion: grade 0 (none); grade 1 (mild); grade 2 (moderate); grade 3 (severe) and Dryness/scaling: grade 0 (none); grade 1 (mild); grade 2 (moderate); grade 3 (severe)

Scratching behaviour: the scratching behaviour was measured before drug treatment and then once a week in the treatment period. Thirty-minute scratching behaviour of the mice was evaluated.

Transepidermal water loss (TEWL): TEWL was measured on individual mice with an evaporimeter (multi-probe Adapter MPAS and TM300 probe, CK electronic GmbH, Germany) before drug treatment and then once a week in the treatment period. The hair on dorsal skins was shaved under light anesthesia before measurement of TEWL. Measurements were performed three times at the same place of dorsal skins at 23° C.±2° C., and 40%±10% relative humidity and the mean value was calculated.

Various grades of dermatitis were observed in all mice of the three groups, and around 8 points (clinical skin scores) were noted in mice when the study was started.

TABLE 13
Clinical severity score

| | Pre | | | | | Day | Day | Day |
|---|---|---|---|---|---|---|---|---|
| Groups | Treatment | Day 2 | Day 4 | Day 6 | Day 8 | 10 | 12 | 14 |
| Group I (Zileuton) | 7.6 | 6.9 | 7.0 | 6.0 | 5.6 | 4.9 | 4.6 | 3.9 |
| Group II (Vehicle) | 8.4 | 9.0 | 9.1 | 9.0 | 9.0 | 8.7 | 8.4 | 8.1 |
| Group III | 8.9 | 8.3 | 7.9 | 7.3 | 6.3 | 5.6 | 5.4 | 5.4 |
| (PROTOPIC ® | | | | | | | | |
| Ointment) | | | | | | | | |

Total skin severity scores of mice in groups of Group I, and Group III were decreased and significantly lower than those of mice in vehicle (Group II) group (Fisher's LSD multiple comparisons). In all groups, clinical skin severity scores tended to be decreased in comparison with pre-treatment, but no statistical significance was observed in vehicle group except on day 14. On day 7 and day 14, the scratching frequency and the total scratching duration were significantly decreased in mice in all groups. In comparison with vehicle group, the scratching frequency was significantly reduced on day 7 and day 14 in Group I and group III, while there was no difference between the total scratching duration in Group I and that in Group II on day 14.

TABLE 14
Scratching frequency (per 30 minutes)

| Groups | Pre-treatment | Day 7 | Day 14 |
|---|---|---|---|
| Group I (Zileuton) | 91.3 | 41.9 | 32.7 |
| Group II (vehicle) | 81.9 | 60.4 | 59.1 |
| Group III (PROTOPIC ®) | 94.4 | 45.9 | 32.6 |

TABLE 15
scratching duration (sec/30 minutes)

| Groups | Pre-treatment | Day 7 | Day 14 |
|---|---|---|---|
| Group I (Zileuton) | 75.4 | 34.6 | 37.3 |
| Group II (vehicle) | 64.0 | 44.2 | 44.7 |
| Group III (PROTOPIC ®) | 78.2 | 33.8 | 21.9 |

The significant TEWL reduction was observed in groups of treatment of Group I, and Group III with treatment for 7 and 14 days compared with pre-treatment. In the group of vehicle treatment (Group II), the TEWL was reduced only on day 14 in comparison with pre-treatment.

TABLE 16
Effect on TEWL

| Groups | Pre-treatment | Day 7 | Day 14 |
|---|---|---|---|
| Group I (Zileuton) | 33.1 | 16.0 | 14.1 |
| Group II (vehicle) | 30.3 | 28.7 | 19.2 |
| Group III (PROTOPIC ®) | 37.3 | 15.9 | 18.1 |

The effect of zileuton was marked in TEWL and was comparable to that of 0.1% PROTOPIC® ointment. All treatments significantly decreased scratching behavior.

### Example 11

### Clinical Study for Evaluation of the Efficacy and Safety of Zileuton Ointment

Study design: The clinical study is designed as a randomized, double-blind, vehicle-controlled, parallel study with three treatment groups: zileuton ointment, 1.25% w/w applied twice daily, zileuton ointment 5% w/w applied twice daily, and vehicle ointment applied twice daily.

Subject visits during this phase are scheduled at baseline (visit 2-day 1), and at the end of week 1 (visit 3-day 8±1 day), end of week 2 (visit 4-day 15±2 days), and end of week 4 (visit 5-day 29±3 days).

At visit 2 (baseline), the subjects will be randomized in a 1:1:1 ratio to one of the three treatment groups. Baseline AD scores (IGA [Investigator's Global Assessment of Disease Severity], EASI [Eczema Area and Severity Index] and SCORAD [SCORing Atopic Dermatitis], and pruritus assessments by Numerical Rating Scale (NRS) will be done. A skin biopsy of 2-3 mm in size will be taken from the selected treatable area. The study product will be applied as a thin layer to all treatable lesions of AD. The subjects will administer the remaining doses twice daily at home for 28 days. The investigational product will not be applied over the biopsy site for 2 days.

At visits 3, 4, and 5, the AD score assessments (IGA, EASI, DLQI, and NRS) will be repeated. SCORAD will be assessed at visit 5. A skin biopsy of 2-3 mm in size (at least 1 cm away from the previous biopsy site) will be taken at visit 4. During the treatment phase, in addition to the investigational products (IP), subjects will be allowed to use a cleanser and moisturizing bath lotion (CETAPHIL®) provided by the Sponsor.

Application site reactions (dryness, burning/stinging, erosion, edema, and pain) will be determined at baseline (visit 2-day 1) and visits 3 to 5 for all treated areas.

Study population: The participant population for the study will include males and/or females aged 12 years or older at the time of screening with moderate to severe AD as per IGA of disease severity.

Inclusion criteria: Male and female subjects must be 12 years of age or older. Subjects must be diagnosed with AD as per revised Hanifin and Rajka criteria. Subjects must have an IGA grade of ‘Moderate (3)’ to ‘Severe (4)’ at baseline. Subjects must have AD lesions affecting ≤20% of the body surface area (BSA) at baseline (subjects will be instructed to avoid applying IP around eyes, mouth, and nostrils). Subjects must be willing to provide a skin biopsy of the target lesion at baseline and end of treatment visits.

Treatment regimen: Subjects will be treated with topical zileuton ointment containing 1.25% w/w or 5% w/w, or the matching topical vehicle ointment, for a maximum of 4 weeks. The total expected study duration is eight weeks, including the screening visit.

Efficacy Assessments:

Investigator's Global Assessment (IGA): The IGA will be conducted on all study visits. It is a 5-point global assessment.

| Category Description | Category | Score |
|---|---|---|
| Minor residual hypopigmentation/hyperpigmentation; | Clear | 0 |
| no erythema or induration/papulation; no | | |
| oozing/crusting | | |
| Trace faint pink erythema, with barely perceptible | Almost | 1 |
| induration/papulation and no oozing/crusting | clear | |
| Faint pink erythema with mild induration/papulation | Mild | 2 |
| and no oozing/crusting | | |
| Pink-red erythema with moderate induration/ | Moderate | 3 |
| papulation with or without oozing/crusting | | |
| Deep or bright red erythema with severe | Severe | 4 |
| induration/papulation and with oozing/crusting | | |

Eczema Area and Severity Index (EASI): The EASI quantifies the severity of a subject's AD based on both severities of lesion clinical signs and the percent of BSA affected. EASI is a composite scoring of the degree of erythema, oedema/papulation, excoriation, and lichenification (each scored separately) for each of four body regions (head/neck, trunk, upper extremities, and lower extremities), with adjustment for the percent of BSA involved for each body region and for the proportion of the body region to the whole body.

| Scratching | Erythema | Edema | Lichenification | |
|---|---|---|---|---|
| (excoriation) | (redness) | (papulation) | (lined skin) | Score |
| No symptoms | No symptoms | No symptoms | No symptoms | 0 |
| Mild | Mild | Mild | Mild | 1 |
| Moderate | Moderate | Moderate | Moderate | 2 |
| Severe | Severe | Severe | Severe | 3 |

Using the table below, give each respective body region a score of between 0 to 6 based on the percentage involvement.

| | % involvement | | | | | | |
|---|---|---|---|---|---|---|---|
| | 0 | 1-9 | 10-29 | 30-49 | 50-69 | 70-89 | 90-100 |
| Region score | 0 | 1 | 2 | 3 | 4 | 5 | 6 |

The final EASI score is the sum of the 4 region scores and will range from 0 to 72.

SCORAD Scoring: The SCORAD scoring will be done at baseline and visit 5. The extent and severity of AD over the body area and the severity of 6 specific symptoms (erythema, edema/papulation, excoriations, lichenification, oozing/crusts, and dryness) are assessed and scored by the investigator. Subjective assessment of itch and sleeplessness is scored by the patient. The SCORAD score is a combined score of body area affected, and investigator and patient symptom scoring, with a maximum of 103.

Pruritis Assessment by NRS: Pruritus scoring by NRS will be done at baseline, day 3 (telephonically), and visits 3, 4, and 5. Pruritus assessment will be done using a subject-rated pruritus score of the severity of pruritus due to AD, suffered in the past 24 hours on an 11-point NRS where 0 is no itch, and 10 is worst itch.

Efficacy Evaluation:

Primary Endpoint: The proportion of subjects with IGA treatment ‘success’, i.e., clear or almost clear; a score of 0 or 1, with at least a two-grade reduction from baseline to week 4 for zileuton ointment 5% w/w compared to vehicle.

Secondary Endpoints:
I. The proportion of subjects with IGA treatment ‘success’, i.e., clear or almost clear; a score of 0 or 1, with at least a two-grade reduction from baseline to week 4 for zileuton ointment 1.25% w/w compared to vehicle
II. The proportion of subjects with at least a two-grade improvement of IGA grade from baseline to week 4 in the zileuton ointment treatment groups compared to vehicle
III. Time to IGA treatment ‘success’ in the zileuton ointment treatment groups compared to the vehicle
IV. Mean change in the BSA involved from baseline to weeks 1, 2, and 4
V. Mean change in the EASI score from baseline to weeks 1, 2, and 4
VI. Mean change in SCORAD from baseline to week 4
VII. Proportion of patients showing a 5-point reduction in DLQI score from baseline to weeks 1, 2, and 4
VIII. Mean change from baseline to day 3 and weeks 1, 2, and 4 in the NRS
IX. Gene expression changes as assessed by skin biopsy from baseline to week 4

1. A topical composition comprising (a) an active agent and (b) an oleaginous base; wherein the oleaginous base comprises a skin penetration enhancer in an amount of less than about 20% w/w based on the total weight of the composition.

2. The topical composition of claim 1, wherein the active agent is selected from the group consisting of zileuton, crisaborole, tacrolimus, doxepin, and combinations thereof.

3. The topical composition of claim 1, wherein the skin penetration enhancer is selected from the group consisting of fatty alcohols, fatty acids, ethers of fatty alcohols, esters of fatty acids, terpenes, vegetable oils, and mixtures thereof.

4. The topical composition of claim 1, wherein the skin penetration enhancer is a fatty acid ester.

5. The topical composition of claim 4, wherein the skin penetration enhancer is selected from the group consisting of disopropyl adipate, diisopropyl sebacate, dibutyl sebacate, isopropyl myristate, isopropyl palmitate, medium chain triglycerides, and methyl propionate.

6. The topical composition of claim 1, wherein the active agent is in a non-solubilized form in the composition.

7. The topical composition of claim 1, wherein the composition is substantially anhydrous.

8. The topical composition of claim 1, wherein the composition is non-foaming and propellant-free.

9. The topical composition of claim 1, wherein the composition is occlusive.

10. A topical composition comprising (a) an active agent, (b) one or more stiffening agent(s), (c) a skin penetration enhancer, (d) an oleaginous vehicle, and (e) a pharmaceutically acceptable excipient(s); wherein the composition comprises the stiffening agent, the skin penetration enhancer, and the oleaginous vehicle in a weight ratio from about 3:2:14 to about 2:1:17.

11. The topical composition of claim 10, wherein the oleaginous vehicle is selected from the group consisting of mineral oil, soft paraffin, hard paraffin, petrolatum, mixture of mineral oil and lanolin alcohols, coconut oil, almond oil, lanolin, mixture of petrolatum and lanolin alcohols, fatty alcohols, vegetable oils, and combinations thereof.

12. The topical composition of claim 10, wherein the skin penetration enhancer is selected from the group consisting of fatty alcohols, fatty acids, ethers of fatty alcohols, esters of fatty acids, terpenes, vegetable oils, and mixtures thereof.

13. The topical composition of claim 10, wherein the oleaginous vehicle has a melting point of more than about 35° C.

14. The topical composition of claim 10, wherein the stiffening agent is selected from the group consisting of white wax, microcrystalline wax, emulsifying wax, colloidal silicon dioxide, and combinations thereof.

15. The topical composition of claim 10, wherein the composition is substantially anhydrous.

16. The topical composition of claim 10, wherein the composition is non-foaming and propellant-free.

17. The topical composition of claim 10, wherein the composition is occlusive.

18. A topical composition comprising (a) an active agent and (b) an oleaginous base; wherein the composition has an oleaginous base in an amount of at least about 60% w/w based on the total weight of the composition, and the composition is free of hydrophilic solvent(s).

19. The topical composition of claim 18, wherein the composition is free of hydrophilic solvents selected from the group consisting of ethanol, isopropyl alcohol, ethylene glycol, polyethylene glycol (2 to 20 monomers), propylene glycol, dipropylene glycol, butylene glycol, pentylene glycol, and hexylene glycol.

20. The topical composition of claim 18, wherein the composition is non-foaming and propellant-free.
